(19) United States
(12) Patent Application Publication
(10) Pub. No.: US 2026/0027327 A1
(43) Pub. Date: Jan. 29, 2026

(54) OTIC DRUG DELIVERY SYSTEM AND METHODS

(71) Applicant: Regeneron Pharmaceuticals, Inc., Tarrytown, NY (US)

(72) Inventors: William A. Duprey, Loudonville, NY (US); Seth Koehler, Brookline, MA (US); Carleton Eduardo Corrales, Newton, MA (US); Connor Block, Medford, MA (US); Bryan Grygus, Clifton Park, NY (US); Igor Drobnjak, Halfmoon, NY (US)

(21) Appl. No.: 19/282,081

(22) Filed: Jul. 28, 2025

Related U.S. Application Data
(60) Provisional application No. 63/676,016, filed on Jul. 26, 2024.
(60) Provisional application No. 63/758,659, filed on Feb. 14, 2025.

(51) Int. Cl. A61M 25/00; A61M 5/145; A61M 5/155; A61M 5/168; A61M 5/46; A61M 25/06

(52) U.S. Cl. CPC A61M 25/0084; A61M 5/145; A61M 5/155; A61M 5/16813; A61M 5/46; A61M 25/0026; A61M 25/0045; A61M 25/005; A61M 25/0068; A61M 25/0097; A61M 25/0662; A61M 2025/0089; A61M 2025/0093; A61M 2025/0681; A61M 2210/0668

(57) ABSTRACT

Disclosed herein are drug delivery devices including catheter assemblies, infusion systems, pre-filled syringes, methods of use, combination products, kits of parts, and drug products with parts lists for independent sourcing, for use in the otic administration of a drug product to a subject in need thereof.

### CROSS REFERENCE TO RELATED APPLICATIONS

The present application claims priority to U.S. Provisional Patent Application No. 63/676,016, filed Jul. 26, 2024, and to U.S. Provisional Patent Application No. 63/758,659, filed Feb. 14, 2025.

### FIELD OF THE DISCLOSURE

Various embodiments of the disclosure relate to a platform for delivery of therapeutic drug products to the middle or inner ear. More particularly, the disclosure relates to an otic drug delivery system, e.g., an intracochlear drug delivery system, and methods for use of the same.

### INTRODUCTION

The human ear anatomy includes three main parts: the outer ear, the middle ear, and the inner ear. The outer ear includes the pinna and the ear canal, and is separated from the middle ear by the tympanic membrane (ear drum). The middle ear includes the ossicles, which are three small bones (malleus, incus, and stapes) that transfer vibrations from sound waves from the ear drum to the inner ear. The eustachian tubes are also part of the middle ear. The inner ear includes the cochlea, which is primarily involved with hearing, and the semi-circular canals, which are primarily involved with balance.

Sound waves cause the ear drum to vibrate, which in turn causes the ossicles to vibrate. These vibrations of the ossicles cause the fluid inside the cochlea to move back and forth. Hair cells in the cochlea convert the fluid movement to signals that are then sent via the auditory nerve to the brain for processing, resulting in the perception of sound.

A common cause of hearing loss is sensorineural hearing loss, a type of hearing loss caused by defects in the cells of the inner ear, such as the cochlear hair cells and cochlear supporting cells, or the neural pathways that project from the inner ear to the brain. Sensorineural hearing loss is often acquired, and has a variety of causes, including acoustic trauma, disease or infection, head trauma, ototoxic drugs, and aging. There are also genetic causes of sensorineural hearing loss, such as mutations in genes involved in the development and function of the inner ear. Mutations in over ninety such genes have been identified, including mutations inherited in an autosomal recessive, autosomal dominant, and X-linked patterns.

For example, in individuals having a mutation in one or more copies of the otoferlin (OTOF) gene, the otoferlin protein is compromised in its ability to release a neural transmitter at the hair cell's synapse. This in turn compromises the hair cells' ability to send signals to the auditory nerve cells, and results in hereditary hearing loss or deafness. Investigational gene therapies are currently in clinical development for the treatment of OTOF-mediated hearing loss, and have demonstrated the feasibility of this therapeutic approach. Such gene therapies may be surgically administered to the patient's cochlea via the round window membrane (RWM). Surgical approaches to the RWM include a trans-canal approach or a trans-mastoid approach. In either surgical approach case, a high level of care is exercised due to the sensitivity and fragility of the ear structures. For example, the introduction of air into the cochlear tubes may cause physical and/or chemical disruption that may damage the hair cells within the organ of corti, destroying the hair cells' ability to mechanically transduce signals. For this reason, it is desirable to avoid introducing air bubbles into the cochlea during administration of a drug product to the cochlea. Additionally, any one or more of excessive movement within the cochlear fluid, introduction of fluid into the cochlea at a high velocity, the use of excessive force to puncture or open the RWM, the over-insertion of a device through the RWM, or any contact between an introduced device and any of the ear structures may also cause damage to ear structures, and are therefore also desirable to avoid during administration.

A platform is therefore needed for the safe and effective delivery of drug products to the middle and inner ear such as, e.g., delivery of gene therapies to the cochlea, while desirably avoiding deleterious effects described herein.

### SUMMARY

A first aspect of the disclosure provides an intracochlear catheter assembly comprising: a sheath having a first lumen axially extending therethrough; and a distal tip adapted for insertion through the round window membrane (RWM), wherein the catheter assembly is configured for intracochlear insertion.

Various embodiments of the intracochlear catheter assembly may additionally or alternatively include an overcoating disposed at least partially over the sheath, such that the sheath forms a liner within the overcoating.

Various embodiments of the intracochlear catheter assembly may additionally or alternatively include one or more of the following features: a rigid layer disposed about the catheter assembly and within the overcoating, the rigid layer being adapted to provide structural support to the catheter, wherein the rigid layer comprises a metal coil, a metal mesh, or a metal braid; a needle integrated into the distal tip and in fluid communication with the first lumen, wherein the needle is adapted for insertion through the RWM and infusion of a fluid; an opening at the distal tip of the sheath, the opening being in fluid communication with the first lumen; or a tapered geometry at the distal tip of the catheter assembly; wherein the distal tip has a shape selected from: blunt, rounded, and pointed.

Various embodiments of the intracochlear catheter assembly may additionally or alternatively include one or more of the following features: a plurality of through-holes in a radially outward facing surface of the overcoating, wherein each through-hole in the plurality of through-holes is in fluid communication with the first lumen, and wherein the distal end of the first lumen is closed; or a connector coupled to a proximal end of the catheter assembly.

Various embodiments of the intracochlear catheter assembly may additionally or alternatively include one or more of the following features: the sheath comprises plastic; the overcoating comprises a depth stop disposed about the overcoating; wherein the depth stop is integrally formed with the overcoating; or the distal tip is adapted for insertion through a hole in the RWM formed by an instrument comprising a notch or a flange adapted to indicate a desired depth of insertion.

Various embodiments of the intracochlear catheter assembly may additionally or alternatively include one or more of the following features: a second lumen disposed therein, the second lumen being substantially parallel with the first lumen, and adapted to receive a guidewire or stylet therein, wherein the second lumen is open to the proximal end of the catheter assembly and is not open to the distal tip of the catheter assembly, wherein the catheter assembly further comprises a tapered portion at the distal tip thereof, and wherein a closed, distal end of the second lumen is disposed proximally relative to a proximal-most end of the tapered portion, such that the second lumen does not extend distally into the tapered portion; a shoulder formed in the overcoating, wherein the shoulder is disposed about a closed distal end of the second lumen, and wherein the shoulder forms a depth stop limiting an extent of insertion of the distal tip of the catheter assembly through the RWM; or a pair of tip members collectively forming the distal tip, each tip member of the pair of tip members being movable between a closed position and an open position, wherein in the closed position, the pair of tip members collectively form a sharp point at a distal end thereof, and the intracochlear catheter assembly is closed at the distal tip, and in the open position, the pair of tip members are adapted to articulate radially outwardly, and the intracochlear catheter assembly is open at the distal tip; and a microcontroller adapted to selectively move the pair of tip members between the closed position and the open position.

Various embodiments of the intracochlear catheter assembly may additionally or alternatively include one or more of the following features: a flexible and collapsable housing disposed over the intracochlear catheter assembly, and movable between a flexible, extended state and a rigid, collapsed state, wherein the housing is configured to contain the intracochlear catheter assembly while allowing the needle to extend distally beyond a distal end of the housing, wherein, in the rigid, collapsed state, the housing is configured to secure a position and an orientation of the needle; an introducer disposed over the intracochlear catheter assembly, the introducer comprising: a sharp distal tip configured to puncture the RWM, and a depth stop disposed proximally relative to the sharp distal tip, the depth stop being configured to limit an extent of insertion of the introducer through the RWM; and a spring disposed within the introducer and adapted to bias the intracochlear catheter assembly in a distal direction.

Various embodiments of the intracochlear catheter assembly may additionally or alternatively include one or more of the following features: a housing disposed over the catheter assembly, the housing including: a distal housing member that is disposed over the distal tip of the intracochlear catheter assembly, and configured to limit distal movement thereof upon contact by the distal housing member with the RWM, wherein the needle is permitted to extend distally beyond the distal housing member; and a proximal housing member coupled to the distal housing member, and comprising a flange at a proximal end thereof, the flange being configured to engage an outer surface of a skull of a patient and thereby limit distal movement of the housing relative to the skull.

Various embodiments of the intracochlear catheter assembly may additionally or alternatively include one or more of the following features: a first sleeve disposed over the overcoating; a pick disposed within a lumen in the first sleeve and configured to translate distally to puncture the RWM upon actuation; a handle coupled to a proximal end of the first sleeve and having an actuator for actuating translation of the pick; a second sleeve disposed over the first sleeve; and a depth stop disposed on the second sleeve and configured to limit a depth to which the intracochlear catheter assembly is insertable into a patient.

A second aspect of the disclosure provides a terminally sterilized, pre-filled syringe for intracochlear injection comprising: a barrel having an outlet at an outlet end and containing a medicament; a needle disposed at the outlet end and in fluid communication with the outlet; a stopper disposed and movable within the barrel; a plunger rod in contact with the stopper; a flange disposed at a proximal end of the barrel; and a spacer disposed about the needle at the outlet end, the spacer being configured to limit a depth to which the needle may be inserted in use.

A third aspect of the disclosure provides an intracochlear infusion system, comprising: an intracochlear catheter assembly comprising: a sheath having a first lumen axially extending therethrough; and a distal tip adapted for insertion through the round window membrane (RWM), wherein the catheter assembly is configured for intracochlear insertion; and a catheter introducer adapted for insertion into the RWM, wherein the catheter introducer is adapted to receive the catheter assembly therein, and wherein the catheter introducer and the catheter assembly are adapted to be removed from the RWM together.

Various embodiments of the intracochlear infusion system may additionally or alternatively include one or more of the following features: an overcoating disposed at least partially over the sheath, such that the sheath acts as a liner within the overcoating; a further sheath disposed over a portion of the catheter assembly; a depth stop coupled to the introducer at a distal end thereof, wherein the distal tip of the assembly extends distally beyond the depth stop; a support structure coupled to the depth stop and extending distally therefrom, wherein the support structure includes a sharp distal end configured to puncture the RWM, and wherein the support structure is configured to partially enclose and support the distal tip of the catheter assembly, wherein the support structure comprises a plurality of blades or prongs circumferentially arranged about the distal tip, or wherein the support structure comprises a sconce disposed about a portion of a circumference of the distal tip, or wherein the support structure comprises a helical structure disposed about the distal tip; a depth stop disposed about the catheter assembly; and a plurality of pinch blocks disposed on an inner surface of the catheter introducer, wherein the plurality of pinch blocks are configured to engage the depth stop on the catheter assembly to secure a position of the catheter assembly within the catheter introducer; or a connector disposed on the depth stop; and an in-lay disposed on a proximal end of the catheter introducer, wherein the in-lay is configured to receive and engage the connector disposed on the depth stop.

A fourth aspect of the disclosure provides an intracochlear infusion system, comprising: an intracochlear catheter assembly comprising: a sheath having a first lumen axially extending therethrough; and a distal tip adapted for insertion through a round window membrane (RWM) of a subject, wherein the catheter assembly is configured for intracochlear insertion; and a feed device disposed on a patient's skin at a location proximate to an incision in the skin through which the RWM is accessible, wherein the feed device is configured to receive the intracochlear catheter therethrough.

Various embodiments of the intracochlear infusion system may additionally or alternatively include one or more of the following features: an overcoating disposed at least partially over the sheath; an adhesive configured to adhere the feed device to the patient's skin; a rotatable dial or knob on the feed device, configured to advance the intracochlear catheter assembly into the RWM, and to retract the intracochlear catheter assembly from the RWM, wherein the rotatable dial or knob is configured to rotate in a first direction to advance the intracochlear catheter assembly into the RWM, and to rotate in a second direction, opposite the first direction, to retract the intracochlear catheter assembly from the RWM, wherein the rotatable dial or knob comprises a micro-adjustment knob, or wherein the feed device comprises a connector at a proximal end thereof, configured to fluidly couple the intracochlear catheter assembly to an infusion pump.

A fifth aspect of the disclosure provides an intracochlear infusion system, comprising: an intracochlear catheter assembly comprising: a sheath having a first lumen axially extending therethrough; and a distal tip adapted for insertion through the round window membrane (RWM), wherein the catheter assembly is configured for intracochlear insertion; and a pump configured to cause fluid to flow through the intracochlear catheter assembly and infuse into a cochlea of a patient.

Various embodiments of the intracochlear infusion system may additionally or alternatively include one or more of the following features: an overcoating disposed at least partially over the sheath; the pump comprises an electroosmotic (EO) pump, a gas-cylinder powered device, an electrochemical cell, or a battery-driven motor.

A sixth aspect of the disclosure provides a kit of parts for performing an intracochlear infusion comprising: an intracochlear catheter assembly comprising: a sheath having a first lumen axially extending therethrough; and a distal tip adapted for insertion through the round window membrane (RWM), wherein the catheter assembly is configured for intracochlear insertion; and an instrument for forming a hole in the RWM, the instrument comprising a depth stop or a depth indicator.

A seventh aspect of the disclosure provides an intracochlear catheter assembly comprising: a microneedle having a sharp distal tip and a first lumen disposed therein; and a catheter having a second lumen, wherein the first lumen and the second lumen are in fluid communication.

Various embodiments of the intracochlear infusion system may additionally or alternatively include one or more of the following features: a hub coupled to a distal end of the catheter and to a proximal end of the microneedle, wherein the hub is formed about the microneedle by an insert-molding process; a third lumen extending longitudinally through the hub, wherein the third lumen is adapted to receive the distal end of the catheter and the proximal end of the microneedle; and adhesive or a weld adapted to affix a position of the distal end of the catheter within the third lumen.

Various embodiments of the intracochlear infusion system may additionally or alternatively include one or more of the following features: a retention feature on the microneedle disposed at or near a proximal end thereof, where the retention feature comprises an outer diameter that is greater than an outer diameter of a shaft of the microneedle, and where the retention feature comprises one of a bead or a flared proximal portion; a proximal end of the hub and a distal end of the catheter being made of same material and abutting one another, and being affixed to one another by a butt weld; a proximal end of the hub being disposed within the second lumen of the catheter; a barb disposed on a radially outward facing surface of the hub, wherein the barb contacts a wall of the second lumen; and a crimp, a collar, or a clamp disposed about an outer surface of the catheter and configured to apply pressure to a fit between the catheter and the hub.

Various embodiments of the intracochlear infusion system may additionally or alternatively include, within the hub, the proximal end of the microneedle being disposed within the second lumen at the distal end of the catheter; a depth stop disposed on a distal end of the hub, wherein the depth stop is distanced from the sharp distal tip of the microneedle by a distance corresponding to a desired insertion depth of the microneedle; the depth stop comprising a first lobe extending radially outward beyond a circumference of the hub; the depth stop further comprising a second lobe extending radially outward beyond the circumference of the hub, wherein the first lobe and the second lobe are separated by cutaway portions, and wherein the cutaway portions are configured to allow a user to visualize the microneedle; the depth stop comprising a third lobe extending radially outward beyond the circumference of the hub, wherein the first lobe, the second lobe, and the third lobe are separated by cutaway portions, and wherein the cutaway portions are configured to allow a user to visualize the microneedle; and the depth stop further comprising a distal surface that is selected from: flat, convex, or angled.

Various embodiments of the intracochlear infusion system may additionally or alternatively include one or more of the following features: the hub comprising an angled portion relative to a longitudinal axis of the hub; the microneedle comprising a silicon needle; a distal end of the catheter being disposed over a proximal end of the microneedle, and the distal end of the catheter forming a depth stop that is distanced from the sharp distal tip of the microneedle by a distance corresponding to a desired insertion depth of the microneedle; the sharp distal tip of the microneedle being selected from: a Quincke needle tip, a Sprotte needle tip, a Whitacre needle tip, a bent needle tip, and a Tuohy needle tip; the catheter further comprising a spring configured to bias the catheter in a distal direction; a visible marking on the catheter configured to indicate to a user one of an insertion depth or a position at which to hold the catheter; and a luer connection at a proximal end of the catheter, configured to securely couple the catheter to a syringe containing a drug product for infusion.

Various embodiments of the kit of parts may additionally or alternatively include one or more of the following features: an overcoating disposed at least partially over the sheath; the instrument comprises a depth stop, and the depth stop comprises a flange; or the instrument comprises a depth indicator, and the depth indicator comprises a notch.

These and other aspects, advantages, and salient features of the disclosure will become apparent from the following detailed description, which, when taken in conjunction with the annexed drawings, disclose embodiments of the invention.

### BRIEF DESCRIPTION OF THE DRAWINGS

The accompanying drawings, which are incorporated into and constitute a part of this specification, illustrate various exemplary embodiments and, together with the description, serve to explain the principles of the disclosed embodiments. The drawings show different aspects of the present disclosure. Where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure.

The embodiments described herein are not limited to any combinations and/or permutations of such aspects and/or embodiments. Moreover, each of the aspects of the described inventions, and/or embodiments thereof, may be employed alone or in combination with one or more of the other aspects of the described inventions and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended reflect or indicate the embodiment(s) is/are “example” embodiment(s).

FIG. 1 illustrates processes in a trans-mastoid surgical approach and administration of a drug product to the inner ear, according to embodiments of the disclosure.

FIG. 2 provides a flow chart including processes in a trans-mastoid surgical approach and administration of a drug product to the cochlea, according to embodiments of the disclosure.

FIG. 3 illustrates administration of a drug product to the cochlea, according to embodiments of the disclosure.

FIG. 4 provides a perspective view of an otic catheter assembly, according to embodiments of the disclosure.

FIGS. 5A-5C provide side views of distal portions of otic catheter assemblies, according to embodiments of the disclosure.

FIGS. 6A-6C provide side views of proximal portions of otic catheter assemblies, according to embodiments of the disclosure.

FIGS. 7A and 7B provide side and cross sectional views, respectively, of an otic catheter assembly in accordance with an embodiment of the disclosure.

FIGS. 8A and 8B provide side and cross sectional views, respectively, of an otic catheter assembly in accordance with an embodiment of the disclosure.

FIGS. 9A-9D provide side views of otic catheter assemblies and instruments for opening the round window membrane in accordance with embodiments of the disclosure.

FIGS. 10A-10B provide side and cross sectional views of otic catheter assemblies in accordance with embodiments of the disclosure.

FIGS. 11A-11B each provide perspective views of otic catheter assemblies in accordance with embodiments of the disclosure.

FIGS. 12A-12B provide side views of otic catheter assemblies in accordance with embodiments of the disclosure.

FIGS. 13A-13E provide side views of otic catheter assemblies in accordance with embodiments of the disclosure.

FIGS. 14A-14B provide side views of an otic catheter assembly and an associated catheter introducer in accordance with embodiments of the disclosure.

FIGS. 15A-15C provide side views of an otic catheter assembly and an associated catheter introducer in accordance with embodiments of the disclosure.

FIGS. 16A-16C provide side views of an otic catheter assembly and collapsable housing in accordance with embodiments of the disclosure.

FIGS. 17A-17B provide side views of an otic catheter assembly and collapsable housing in accordance with embodiments of the disclosure.

FIGS. 18A-18D provide side views of a needle having an otic catheter assembly disposed therein in accordance with embodiments of the disclosure.

FIG. 19 provides a side view of an otic catheter assembly in accordance with embodiments of the disclosure.

FIGS. 20A-20B provide side views of a pre-filled syringe for otic administration of a drug product in accordance with embodiments of the disclosure.

FIGS. 21A-21B provide side views of an otic catheter assembly and associated feed device in accordance with embodiments of the disclosure.

FIGS. 22A, 22B, and 22C provide side, top, and side views, respectively, of an otic catheter assembly and associated feed device in accordance with embodiments of the disclosure.

FIG. 23 provides a side view of an otic catheter assembly in accordance with embodiments of the disclosure.

FIGS. 24-26 provide side views of otic catheter assemblies having a catheter, a hub, and a microneedle in accordance with embodiments of the disclosure.

FIG. 27 provides a cross-sectional view of a portion of an otic catheter assembly having a depth stop in accordance with embodiments of the disclosure.

FIGS. 28-29 provide a cross-sectional view and a perspective view, respectively, of a portion of an otic catheter assembly having a depth stop in accordance with embodiments of the disclosure.

FIGS. 30-31 provide a cross-sectional view and a perspective view, respectively, of a portion of an otic catheter assembly having a depth stop in accordance with embodiments of the disclosure.

FIG. 32 provides a cross-sectional view of a portion of an otic catheter assembly having a depth stop in accordance with embodiments of the disclosure.

FIG. 33 provides a perspective view of a portion of an otic catheter assembly having a depth stop in accordance with embodiments of the disclosure.

FIG. 34A provides a perspective view of a portion of an otic catheter assembly including a depth stop as shown in FIG. 31, in accordance with embodiments of the disclosure.

FIGS. 34B and 34C provide bottom and side views, respectively, of the distal surface of a depth stop corresponding to distal face 710 of FIG. 34A in accordance with embodiments of the disclosure.

FIGS. 34D and 34E provide bottom and side views, respectively, of the distal surface of a depth stop corresponding to distal face 710 of FIG. 34A in accordance with embodiments of the disclosure.

FIG. 34F provides a side of the distal surface of a depth stop corresponding to distal face 710 of FIG. 34A in accordance with embodiments of the disclosure.

FIGS. 35-36 provide perspective views of otic catheter assemblies, each having a catheter, a hub, and a microneedle in accordance with embodiments of the disclosure.

FIGS. 37-38 provide side views of otic catheter assemblies having a catheter, a hub, and a microneedle in accordance with embodiments of the disclosure.

FIGS. 39-40 provide side views of otic catheter assemblies having a catheter and a microneedle in accordance with embodiments of the disclosure.

FIG. 41 provides a cross-sectional view of an otic catheter assembly having a catheter, a hub, and a microneedle in accordance with embodiments of the disclosure.

FIGS. 42-43 provide side views of otic catheter assemblies having a catheter and a microneedle in accordance with embodiments of the disclosure.

FIGS. 44-49 provide side views of aspects of needles which may be incorporated into otic catheter assemblies in accordance with embodiments of the disclosure.

FIGS. 50-53 provide side views of otic catheter assemblies having a catheter and a microneedle in accordance with embodiments of the disclosure.

FIGS. 54-57 provide side views of portions of otic catheter assemblies having a shape memory feature in accordance with embodiments of the disclosure.

FIGS. 58-62 provide side views of portions of otic catheter assemblies having a wing or flag feature adapted for forceps manipulation, in accordance with embodiments of the disclosure.

FIGS. 63-64 provide side views of portions of otic catheter assemblies having holding features in accordance with embodiments of the disclosure.

FIGS. 65-66 provide portions of a cross-sectional view and a perspective view, respectively, of an otic catheter assembly having holding features in accordance with embodiments of the disclosure.

FIGS. 67-68 provide side views of portions of otic catheter assemblies having holding features in accordance with embodiments of the disclosure.

FIG. 69 provides a side view of a portion of an otic catheter assembly having a silicon needle in accordance with embodiments of the disclosure.

FIG. 70 provides a perspective view of a catheter support device adapted for use with catheter in accordance with embodiments of the disclosure.

FIGS. 71-74 provide side views of portions of otic catheter assemblies, each including a priming window, in accordance with embodiments of the disclosure.

It is noted that the drawings of the disclosure are not necessarily to scale.

### DETAILED DESCRIPTION

Embodiments of the present disclosure relate to the administration of therapeutic drug products (DPs) to the middle or inner ear including, e.g., intratympanic, transtympanic, or intracochlear delivery. Various embodiments described herein include delivery devices, e.g., infusion systems, catheter assemblies, pre-filled syringes, and so on, kits of parts adapted for administration of medications to the middle or inner ear, e.g., via infusion or injection, lists of parts for independent sourcing, combination products, as well as delivery methods for performing the same.

As used herein, “administration” refers to providing or giving to a subject a therapeutic agent (e.g., a composition containing a drug product (DP) as described elsewhere herein). Exemplary routes of administration are described herein below.

As used herein, the term “pharmaceutical composition” refers to a mixture containing a therapeutic agent, optionally in combination with one or more pharmaceutically acceptable excipients, diluents, and/or carriers, to be administered to a subject, such as a mammal, e.g., a human, in order to prevent, treat or control a particular disease or condition affecting or that may affect the subject.

As used herein, the terms “effective amount,” “effective dose,” “therapeutically effective amount,” “therapeutically effective dose,” and a “sufficient amount” (or “sufficient dose”) of a drug product, e.g., composition, vector construct, or viral vector described herein refer to a quantity sufficient to, when administered to the subject in need thereof, including a mammal, for example a human, effect beneficial or desired results, including clinical results, and, as such, an “effective amount” or synonym thereof depends upon the context in which it is being applied. For example, in the context of treating sensorineural hearing loss, it is an amount of the drug product, e.g., composition, vector construct, or viral vector sufficient to achieve a treatment response as compared to the response obtained without administration of the drug product. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art.

As used herein, the term “subject” refers to an animal on whom the process or procedure is to be performed, i.e., to whom the infusion or injection may be administered. The animal may be a mammal, and may more particularly be a human, or may be a domesticated animal such as a dog, cat, ferret, hamster, rabbit, mouse, rat, cattle, sheep, pig, goat, horse, donkey, camel, and so on. Other terms such as, e.g., “patient” or “individual” may be used synonymously. A subject may further include an individual suffering from a disease or disorder ameliorated by the administration of an infused or injected drug product (DP) to the cochlea. Such a patient may be considered to be a patient or subject in need of such treatment. The terms “clinician,” “practitioner,” “surgeon,” or “user” are used interchangeably to refer to the individual performing the administration of the infusion or injection on the subject as described herein.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items.

The term “distal end” or any variation thereof, refers to the portion of a device farthest from an operator of the device during a process such as, e.g., an injection operation. For example, the distal end of a syringe would be the needle end of the syringe, and the distal end of a needle would be the sharp tip. Conversely, the term “proximal end,” or any variation thereof, refers to the portion of the device closest to the operator of the device in use. For example, the proximal end of a syringe would be the plunger end of the syringe, and the proximal end of a needle would be the end coupled to a needle hub. Further, as used herein, the terms “about,” “substantially,” and “approximately” generally mean±10% of the indicated value.

### Drug Products and Therapeutic Uses

As noted, embodiments of the present disclosure relate to delivery devices, kits of parts, lists of parts for independent sourcing, combination products, and delivery methods for administration of therapeutic drug products (DPs) to the middle car or inner ear including, e.g., intratympanic, transtympanic, and intracochlear administration. In some embodiments, the delivery devices, kits of parts, lists of parts for independent sourcing, combination products, and delivery methods include those useful for the treatment of sensorineural hearing loss and auditory neuropathy, and in particular embodiments, for the treatment of hearing deficits due to variants in otoferlin (OTOF), e.g., biallelic otoferlin (OTOF) mutations and profound sensorineural hearing loss; hearing loss due to mutations in stereocilin (STRC); hearing loss due to mutations in solute carrier (SLC26A4), which encodes Pendrin, including, e.g., pendrin-related hearing loss, vestibular dysfunction (e.g., pendrin-related vestibular dysfunction or vestibular dysfunction associated with damage to or loss of vestibular hair cells), and Meniere's disease (e.g., hearing loss, tinnitus, or vestibular dysfunction associated with Meniere's disease); and treatment of GJB2-related hearing loss such as, e.g., GJB2-related early presbycusis, pediatric progressive GJB2-related hearing loss, and for congenital GJB2-related hearing loss; prevention of hearing loss in a subject receiving a platinum-based anti-neoplastic agent, e.g., cisplatin-induced ototoxicity; treatment of Sudden Sensorineural Hearing Loss (SSNHL); preservation of hearing after cochlear implantation; treatment of Usher Syndrome type 3A; treatment of vestibular schwannoma (VS), or acoustic neuroma. Subjects may be human, and may be any age, e.g., infant, juvenile, or adult.

In further embodiments, the delivery devices, kits of parts, lists of parts for independent sourcing, combination products, and delivery methods may be useful for the delivery of therapeutic DPs to the middle or inner ear including gene therapies such, e.g., adeno-, adenoassociated-, and lentiviral vector-based therapies, and small molecule-based therapies. Non-limiting examples of such DPs include adeno-associated (AAV)-based gene therapies for the treatment of inner ear hearing loss including, e.g., DB-OTO (Decibel Therapeutics, Inc., Boston, MA, US, described in US 20240309399 A1), therapies including polynucleotides and vectors capable of promoting expression of GJB2 (Decibel Therapeutics, Inc., Boston, MA, US, described in WO/2023/150689 A2), therapies including polynucleotides and vector systems capable of promoting expression of stereocilin (Decibel Therapeutics, Inc., Boston, MA, US, described in WO 2022/235933 A2), therapies including polynucleotides and vectors capable of increasing expression of the SLC26A4 gene (Decibel Therapeutics, Inc., Boston, MA, US, described in WO 2024/031040 A2), GJB2-GT (Sensorion SA, Montpellier, FR), OTOF-GT (SENS-501) (Sensorion SA, Montpellier, FR), AK-OTOF (Akouos, Inc., Boston, MA, US); AK-CLRN1 (Akouos, Inc., Boston, MA, US), and AK-antiVEGF (Akouos, Inc., Boston, MA, US); and small molecule drug products such as, e.g., steroids, DB-020, (Decibel Therapeutics, Inc., Boston, MA, described in WO 2020/243536 A1 and U.S. Pat. No. 10,709,732 B2) for the treatment of middle ear hearing loss; and R-azasetron besylate (SENS-401) (Sensorion SA, Montpellier, FR), for treatment of Sudden Sensorineural Hearing Loss (SSNHL), prevention of cisplatin-induced ototoxicity, and residual hearing preservation after cochlear implantation. Each of the foregoing US and PCT published patent applications and patents, namely US 20240309399 A1, U.S. Pat. No. 10,709,732 B2, WO 2022/235933 A2, WO 2024/031040 A2, WO/2023/150689 A2, and WO 2020/243536 A1, is incorporated by reference as though fully set forth herein.

In particular examples, the delivery devices, kits of parts, lists of parts for independent sourcing, combination products, and delivery methods may be useful for otic delivery, e.g., intracochlear delivery of, e.g., DB-OTO, which may be in the form of an aqueous suspension comprising an OTOF dual vector system, 10 mM sodium phosphate or disodium phosphate, 180 mM sodium chloride, 5% (w/v) sucrose, and 0.001% (w/v) poloxamer 188 at a pH of 7.4, wherein the OTOF dual vector system comprises: a first AAV vector comprising a first ITR sequence; a Myosin 15 (Myo15) promoter operably linked to a first coding polynucleotide that encodes an N-terminal portion of an OTOF isoform 5 protein; a splice donor sequence positioned 3′ of the first coding polynucleotide; a recombinogenic region positioned 3′ of the splice donor sequence; and a second ITR sequence; and a second AAV vector comprising a first ITR sequence; a second recombinogenic region; a splice acceptor sequence positioned 3′ of the second recombinogenic region; a second coding polynucleotide that encodes a C-terminal portion of the OTOF isoform 5 protein positioned 3′ of the splice acceptor sequence; a poly(A) sequence positioned 3′ of the second coding polynucleotide; and a second ITR sequence; wherein the first coding polynucleotide and the second coding polynucleotide that encode the OTOF isoform 5 protein do not overlap, and wherein neither the first nor second AAV vector encodes the full-length OTOF isoform 5 protein, as described in US 2024/0309399 A1, previously incorporated by reference.

### Delivery Method

The surgical approaches and delivery methods described herein may be useful for the administration of drug products into the middle or inner ear for the treatment of diseases and disorders of the car such as those described herein above. In an exemplary delivery method, a drug product is delivered to the inner ear (e.g., into the perilymph) of a human subject, e.g., for the treatment of sensorineural hearing loss such as genetic hearing loss or acquired hearing loss, or for the treatment of vestibular dysfunction, such as vertigo, dizziness, imbalance (e.g., loss of balance or a balance disorder), oscillopsia, or bilateral vestibulopathy).

Various surgical approaches may be used to access the middle and/or inner ear for delivery of drug products. In certain embodiments, a trans-canal approach may be used, in which the middle and/or inner ear is accessed via the ear canal. A surgical device may be used in combination with an endoscope to visualize the target surgical area, e.g., the middle or inner ear. To access the inner ear, e.g., the cochlea and semi-circular canals, the tympanic membrane may be temporarily removed or opened, e.g. via a “flap” technique, a fenestration hole may be drilled on the stapes, and the surgical device may access the cochlea via the round window membrane (RWM) for delivery of drug product to the interior of the cochlea. Access to the middle ear may be gained via the trans-canal approach by temporarily removing or opening the tympanic membrane as described above. This opening may used to insert a delivery device, e.g., a catheter, a needle, a microneedle, a port, e.g., with a wicking device, or other delivery device into or through the tympanic membrane for administration of drug product directly into the middle ear space.

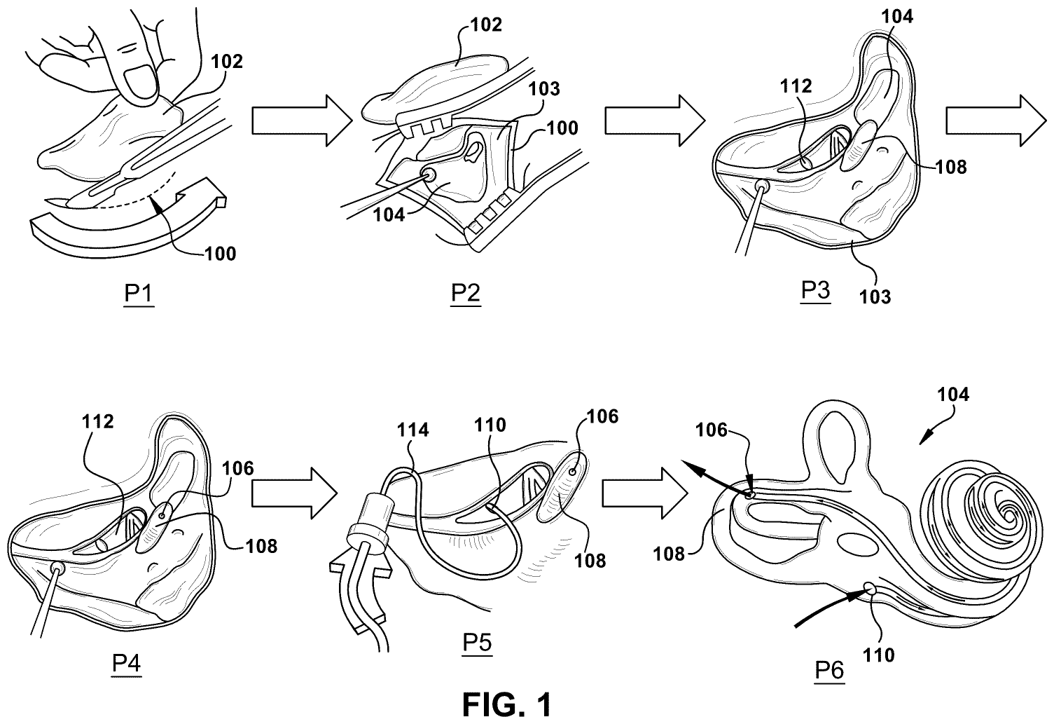

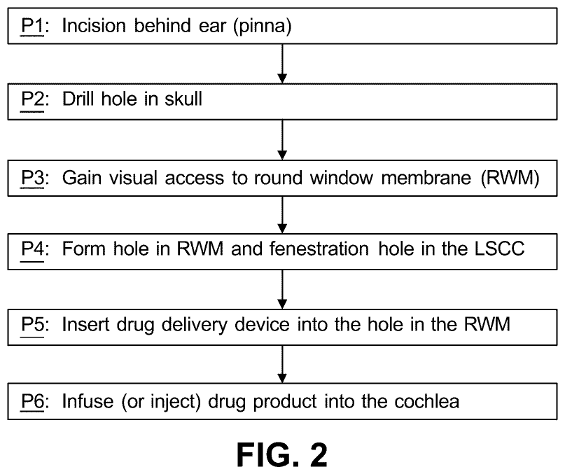

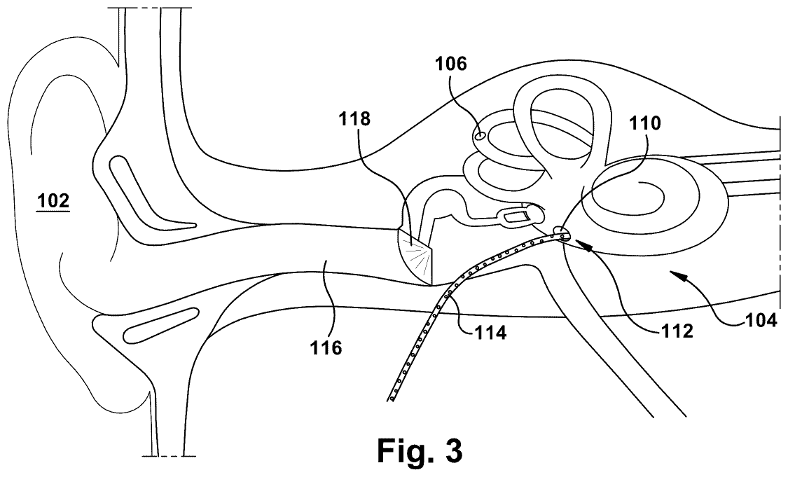

The trans-mastoid approach provides an alternate surgical approach to the middle or inner ear. Turning to the figures, FIGS. 1-3 illustrate through drawings (FIGS. 1 and 3) and a flow chart (FIG. 2), processes P1-P3 in an exemplary trans-mastoid surgical approach, and processes P4-P6 for drug product administration which, in accordance with embodiments described herein, enable delivery of a drug product to the cochlea of a subject in need of such treatment.

Referring to FIGS. 1-2, in process P1, the clinician makes a post-auricular skin incision 100 behind the pinna 102 of the subject's ear. In process P2, the clinician drills an access hole 103 in the subject's skull behind the ear and through the incision 100. At process P3, the clinician gains visual access to the cochlea 104 through the hole 103. In process P4, a fenestration hole 106 is drilled on the lateral semicircular canal (LSCC) 108. The fenestration hole 106 is provided for release of fluid and/or pressure during the drug administration processes. A hole 110 is also created in the round window membrane (RWM) 112, through which a delivery device 114 may be inserted. In process P5, a delivery device 114 such as, e.g., a catheter may be inserted, e.g., threaded, into the cochlea 104, e.g., the scala tympani through the hole 110. In process P6, the drug product may be administered, e.g., infused or injected using drug delivery device 114, into the cochlea 104 via the hole 110 in the RWM 112. Perilymph, excess drug product, or a mixture thereof may exit the cochlea 104 via the fenestration hole 106 in the LSCC 108, thereby avoiding any build-up of fluid and/or pressure in the cochlea 104 which may otherwise be damaging to the ear anatomy. In certain embodiments, the volume of drug product administered may be in excess of the volume of perilymph contained in the subject's cochlea prior to the procedure, such that all fluid in the cochlea 104 is replaced with drug product during the administration processes. The trans-mastoid approach may also be used to access the middle ear, by performing processes P1 and P2, and accessing the mastoid air cells and middle ear.

In contrast with a trans-canal approach to the middle or inner ear, the trans-mastoid approach described herein does not pass through the ear canal 116 or tympanic membrane 118 (FIG. 3), thereby avoiding risk of harm to these structures throughout the processes. However, as noted above, a trans-canal approach to the middle or inner ear may be used in certain embodiments instead of the trans-mastoid approach. The particular approach used may be selected based on any of a number of factors including, e.g., clinician preference or judgment, subject anatomy, or other clinical factors and/or relevant features.

Prior to, in parallel with, or after accessing the target administration region of the ear via either the trans-canal or trans-mastoid approach, the delivery device 114 may be prepared to administer the drug product. An extraction needle may be removed from sterile packaging, and placed on a syringe such as, e.g., a luer lock syringe. The extraction needle and syringe may be used to extract the drug product from a vial in which it is packaged. The extraction needle may then be removed from the syringe, and the syringe may be primed. The syringe may then be coupled, e.g., using a luer connection thereof, to a complementary fitting (e.g., luer connection) on a catheter. The catheter may then be primed with drug product. The syringe may be placed in a syringe pump for use.

An opening 110 may then be created in the RWM 112, and the distal tip of the delivery device 114 may be inserted therethrough. In various embodiments, the opening 110 in the RWM 112 may be created prior to or simultaneously with (e.g., by) insertion of the distal tip of the delivery device 114 into the RWM 112. With the delivery device 114 in position within the cochlea, the syringe pump may be actuated to administer the drug product. The drug product may be infused into the cochlea at a rate of, e.g., 0.5 mL/hr. to 1 mL/hr, e.g., 0.6 mL/hr. to 1 mL/hr, 0.7 mL/hr. to 1 mL/hr, 0.8 mL/hr. to 1 mL/hr, or 0.9 mL/hr. to 1 mL/hr., or 0.72 mL/hr to about 0.96 mL/hr (i.e., 0.24 mL over 15-20 mins.), to avoid damaging fragile structures in the cochlea. The drug product may at least partially, and in certain embodiments fully replace the perilymph, and excess drug product may flow out of the cochlea via fenestration hole 106 such that buildup of fluid and pressure in the cochlea are avoided during the administration process. Following completion of the infusion, the delivery device 114 may be withdrawn from the RWM, the opening 110 in the RWM 112 is closed, the fenestration hole 106 may be patched, and the surgical incision closed.

### Delivery Devices

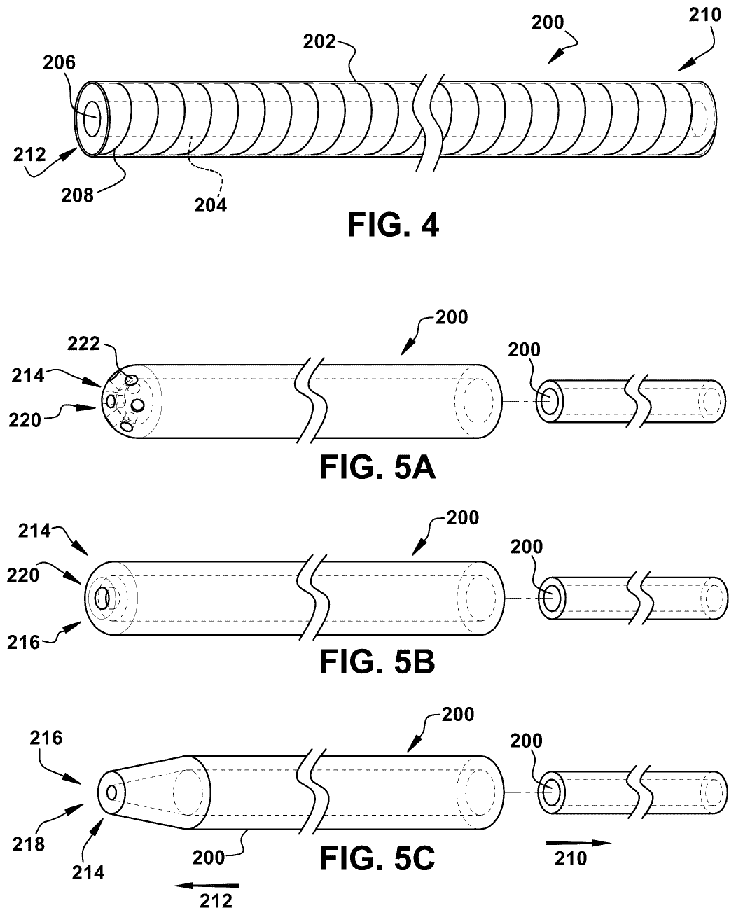

In accordance with the embodiments of FIGS. 4 through 6A-6C (and others), the delivery device 114 may be an otic catheter assembly 200, e.g., an intracochlear catheter assembly. As used herein, the term “catheter” is used to refer to any type of tube adapted for insertion into a body cavity, duct, vessel, or tissue, to permit drainage of fluid, administration of fluid or gas, access by surgical instruments, and/or to perform a wide variety of procedures including, but not limited to intracochlear administration of therapeutic drug products. While described herein in certain examples as intracochlear catheter assemblies, the delivery devices described herein may additionally or alternatively be equally useful for administration of drug products to other target sites within the middle and/or inner ear, e.g., intratympanic or transtympanic administration. As shown in FIG. 4, the otic catheter assembly 200 may comprise one or more sub-assemblies. The sub-assemblies include, e.g., a sheath or liner 204 having a first lumen 206 extending axially therethrough, a jacket or overcoating 202, and a rigid layer 208 adapted to provide rigidity to the catheter assembly. In certain embodiments, the catheter assembly may include only the sheath 204 sub-assembly, e.g., the liner or sheath 204 sub-assembly may itself be the catheter assembly 200. The first lumen 206, disposed within the sheath 204, may be adapted to carry drug product through the catheter assembly 200. In other embodiments, an overcoating 202 may be disposed at least partially over the sheath 204 such that the sheath 204 acts as a liner within the overcoating 202. The liner 204 may limit or prevent contact between the drug product conducted through the first lumen 206 and other sub-assemblies such as, e.g., the overcoating 202. In certain further embodiments, a rigid layer 208 may be disposed about the liner 204 and within the overcoating 202. The rigid layer 208 may be adapted to provide structural support to the otic catheter assembly 200. In other embodiments, the overcoating 202 and the liner 204 may be present without the rigid layer 208. Additionally, in any of the foregoing embodiments, the liner or sheath 204 may contain a first lumen 206 and optionally a second lumen, as described further herein below.

In certain embodiments, the overcoating 202 and the liner 204 may be formed by extrusion, and may be made of plastic. As noted, the liner 204 may be adapted to deliver a drug product therethrough, and to prevent the drug product from contacting the overcoating 202 or the rigid layer 208 when present. The liner or sheath 204 may be flexible on its own, but may gain rigidity through combination with other sub-assemblies of the otic catheter assembly 200 as described herein, e.g., rigid layer 208. The rigid layer 208 may include, e.g., coil, a spring, a mesh, or a braid made of metal such as, e.g., stainless steel or another non-metal material.

As shown in FIGS. 5A-5C, various configurations are provided for the distal end 212 of the catheter assembly 200 including a distal tip 214 that is adapted for insertion through a hole 110 in the RWM 112 as described above relative to FIGS. 1-3 for otic, e.g. intracochlear drug administration.

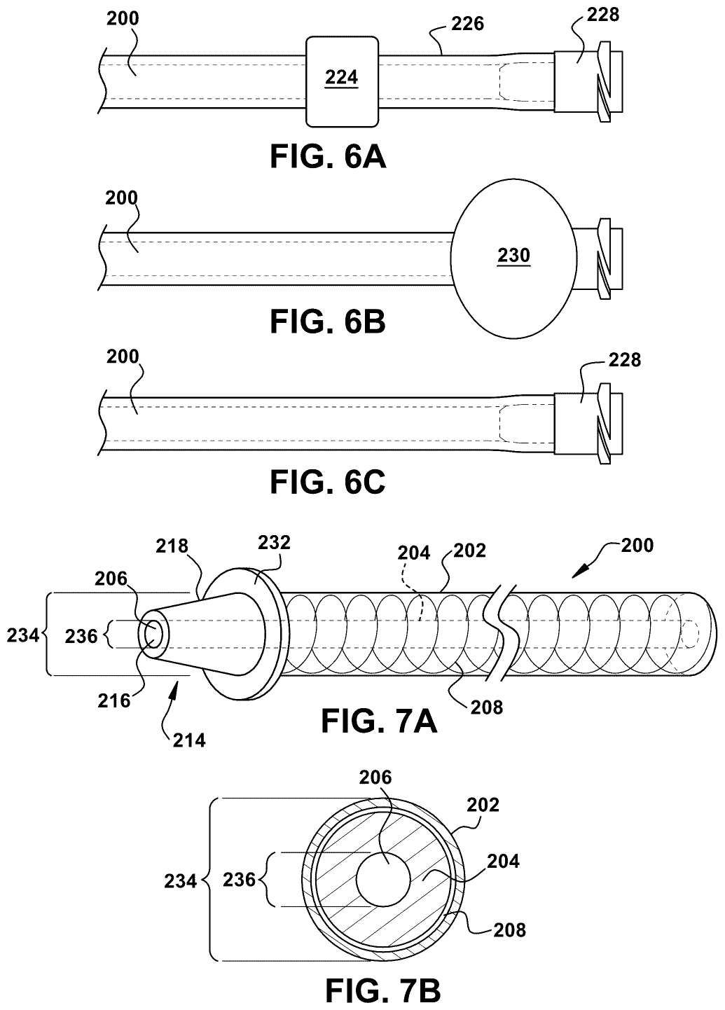

Various configurations are also provided for the proximal end 210. With reference to FIGS. 6A-6C, the otic catheter assembly 200 may further include a connector coupled to a proximal end 210 of the catheter assembly 200. The presence of the connector may facilitate fluid connection between the proximal end 210 of the otic catheter assembly 200 and, e.g., a syringe containing the drug product. Various connectors may be used, including, without limitation, luer slip connectors, luer lock connectors, neuraxial connectors, and so on. One exemplary connector configuration, shown in FIG. 6A, may include, e.g., a connector hub 224 coupled to an extension 226, which may in turn be connected to a luer lock hub 228. Another exemplary connector configuration, shown in FIG. 6B, may include, e.g., a connector attachment 230. In still another exemplary connector configuration, shown in FIG. 6C, a luer lock hub 228 may be directly coupled to the proximal end 210 of the otic catheter assembly 200. These exemplary configurations, and others, may be combined with various other features and embodiments described herein, as will be appreciated by one of skill in the art.

Referring back to FIG. 5A, in certain embodiments, the otic catheter assembly 200 may include a blunt or rounded distal tip 214. The blunt distal tip 214 may be closed, i.e., it may not include an opening at the distal end thereof. Rather, the distal tip may include a plurality of through-holes 222 extending radially outward and being spaced about the distal end of the catheter assembly 200. The through-holes 222 extend through a full thickness of the wall of the catheter assembly 200, placing the first lumen 206 in fluid connection with the exterior of the catheter assembly 200. In use, the through-holes 222 may permit the infusion of fluids from the first lumen 206 of the catheter assembly 200 into a subject, e.g., into a cochlea of the subject. The plurality of through-holes 222, each conducting infused drug product in a different direction and in a relatively small fraction of the total volume of drug product to be infused, limit the pressure and volume introduced into any one location, e.g., within the subject's cochlea. The catheter assembly 200, including the distal tip 214 having through-holes 222, may be manufactured using thermoforming processes.

With reference to, e.g., FIGS. 5B, 5C, 7A, and 7B, other embodiments are shown, in which the otic catheter assembly 200 may include an opening 216 at the distal tip 214 of the catheter assembly 200. The opening 216 is in fluid communication with the first lumen 206 in the catheter assembly 200, e.g., in liner or sheath 204 (as shown in FIG. 7A and 7B).

As best shown in FIGS. 5C and 7A, the catheter assembly 200 may include a tapered geometry 218 at the distal tip 214 of the catheter assembly 200. The tapered geometry 218 may include a segment of the catheter assembly 200 located near the distal tip 214 that gradually and smoothly tapers from a first, larger outer diameter 234 at the proximal end of the segment, to a second, smaller diameter 236 at the distal end of the segment, such that the distal tip 214 of the catheter assembly 200 has the second, smaller diameter 236. Referring back to FIG. 5B, the distal tip 214 may also be blunt or rounded in shape 220.

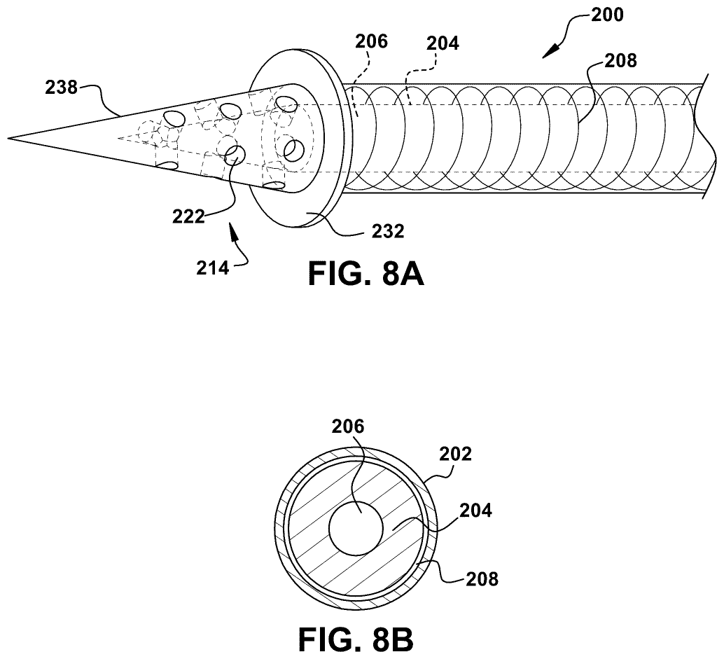

In certain embodiments, as shown in FIGS. 8A and 8B, the catheter assembly 200 may include a needle such as, e.g., a microneedle 238 integrated into the distal tip 214. As used herein, the term “microneedle” refers to needles of any material or fabrication method sized 28 gauge or smaller, or corresponding to a size of 28 gauge or smaller. For example, the microneedle 238 may be, or may correspond in size to, a 28 gauge needle, 29 gauge needle, 30 gauge needle, 31 gauge needle, 32-gauge needle, and so on. The microneedle 238 may be in fluid communication with the lumen containing the drug product, e.g., the first lumen 206. In certain embodiments, e.g., those embodiments including a rigid layer 208, the microneedle 238 and the rigid layer 208 may be made of metal, and the microneedle 238 may be coupled to the rigid layer 208 by welding, by other means of affixation, or may be integrally formed therewith. In other embodiments, the microneedle 238 may be made of the same material as, e.g., the overcoating 202, and may be formed as an extension thereof. In such embodiments, the microneedle 238 may be made of, e.g., plastic. The microneedle 238 may be adapted for insertion through the RWM and infusion of a fluid. The tip of the microneedle 238 may include a plurality of through-holes 222 disposed on a radially outward facing thereof. Each through-hole 222 in the plurality of through-holes 222 may be in fluid communication with the lumen of the catheter assembly 200 containing the drug product, e.g., the first lumen 206, such that upon insertion, the drug product may be infused from the first lumen 206 into the cochlea via the through-holes 222 in a manner similar to that described above relative to FIG. 5A. In such embodiments, as shown in FIG. 8A, the distal end of the lumen, e.g., of the first lumen 206 may be closed, meaning that there is no opening 216 at the distal end thereof as shown in the embodiment of FIG. 7A. The microneedle 238 may have any desired tip geometry, e.g., beveled tip (exemplified in FIGS. 12A-12B), cone (exemplified in FIG. 8A), tapered cone, pyramidal, and so on.

With reference to FIGS. 7A and 8A, the otic catheter assembly 200 may further include a depth stop 232 disposed about the catheter assembly 200, e.g., about the overcoating 202. The depth stop 232 may be axially positioned such that the depth stop 232 is disposed proximally relative to the opening 216 and the tapered geometry segment 218 (FIG. 7A) or proximally relative to through-holes 222 (FIG. 8A) as applicable. The depth stop 232 may further extend circumferentially about and radially outward from the radially outward facing surface of the catheter assembly 200. In certain examples, the depth stop 232 may extend 360° around the circumference of the catheter assembly 200. The depth stop 232 may be integrally formed with the overcoating 202, or may be a separately fabricated component that is coupled thereto.

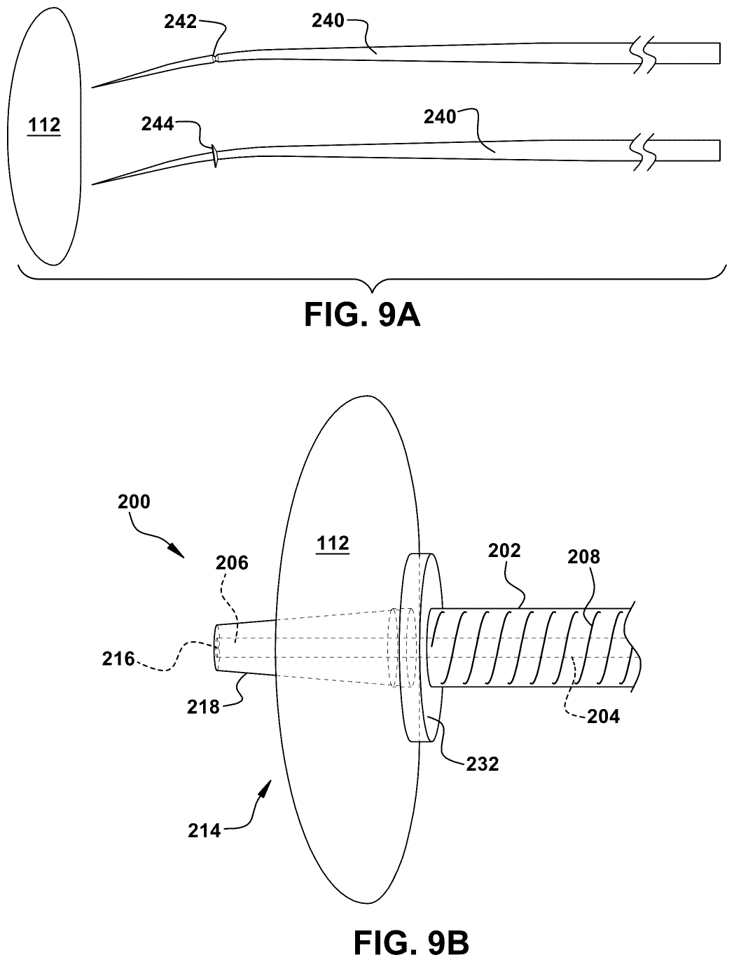

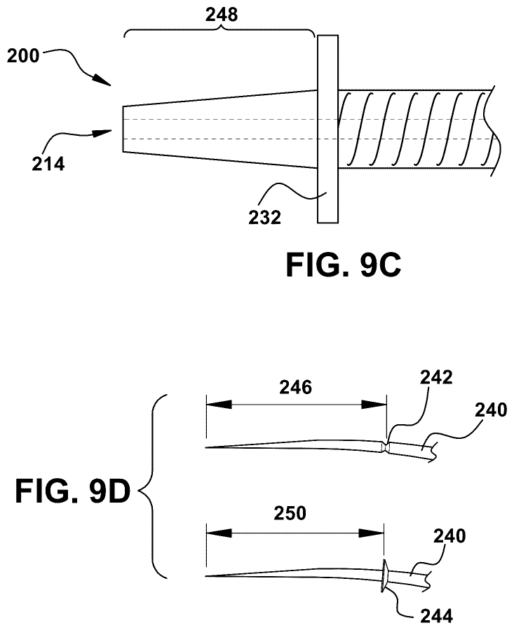

Referring collectively to FIGS. 9A-9D, the distal tip 214 of the catheter assembly 200 (FIGS. 9B, 9C) may be adapted for insertion through a hole in the RWM 112 that is formed by an instrument such as, e.g., a Rosen pick 240 that includes a notch 242 or a flange 244 adapted to indicate a desired depth of insertion (FIGS. 9A, 9D). Where the Rosen pick 240 includes a notch 242, the distance 246 from the distal tip of the Rosen pick 240 to the notch 242 corresponds to the desired depth 248 to which the catheter assembly 200 is inserted into the RWM in use (see FIG. 9C). This may also correspond to the distance 248 from the distal tip of the catheter assembly 200 to a depth stop 232. Referring to FIGS. 9A and 9D, where the Rosen pick 240 includes a flange 244, the distance 250 from the distal tip of the Rosen pick 240 to the flange 244 corresponds to the desired depth 248 to which the catheter assembly 200 is inserted into the RWM in use. This may also correspond to the distance 248 from the distal tip of the catheter assembly 200 to a depth stop 232 (shown in FIG. 9C). Additionally, the outer diameter of the Rosen pick 240 immediately distal of the notch 242 or flange 244 (as applicable) may correspond or substantially correspond to the outer diameter of the catheter assembly 200 immediately distal of the depth stop 232, such that the opening in the RWM created by the Rosen pick 240 provides an exact or close fit with the catheter assembly 200 in use.

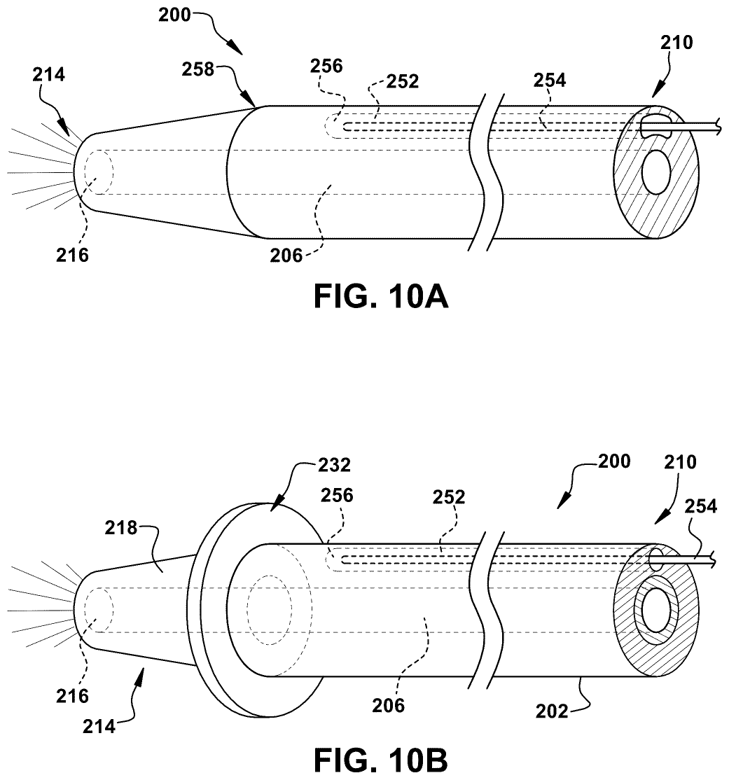

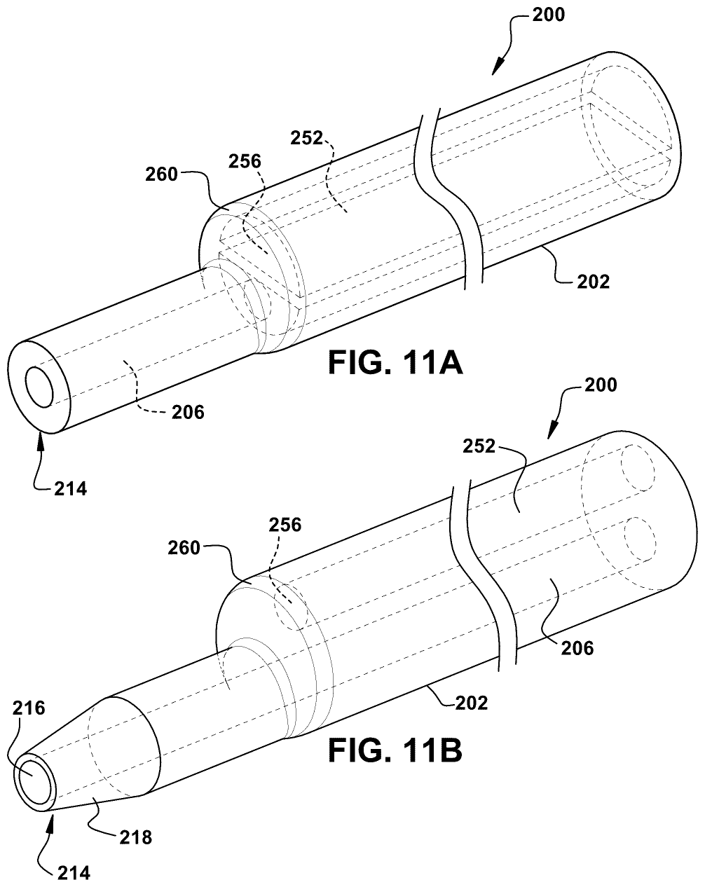

Turning next to FIGS. 10A-10B and 11A-11B, the catheter assembly 200 may further include a double lumen catheter. For example, in addition to the first lumen 206 disposed within the catheter assembly 200, a second lumen 252 may be disposed within the catheter assembly 200. In certain embodiments, the second lumen 252 may be, e.g., within an overcoating 202, while in other embodiments, the catheter assembly 200 may be made of a single layer, e.g. sheath 204, having both of the first lumen 206 and the second lumen 252 disposed therein. Such a catheter assembly 200 may be formed by, e.g., extrusion. In either case, the second lumen 252 may be substantially parallel to the first lumen 206, and may adapted to receive a guidewire or stylet 254 therein from a proximal end thereof. As shown in, e.g., FIGS. 10A-10B, the second lumen 252 may be open to the proximal end 210 of the catheter assembly 200, so as to receive a stylet or guidewire 254 therein, while not being open to the distal tip 214 of the catheter assembly 200. Instead, the second lumen 252 may terminate at a closed, distal end 256. Additionally, double lumen (i.e., having first lumen 206 and second lumen 252) embodiments of the catheter assembly 200 may further include a tapered geometry segment 218 at the distal tip 214, about the first lumen 206. The closed, distal end 256 of the second lumen 252 may be disposed proximally relative to a proximal-most end 258 (FIG. 10A) of the tapered portion 218, such that the second lumen 252 does not extend distally into the tapered portion 218. Thus, the circumference of the catheter assembly 200 accommodates the dual lumens 206, 252 extending distally along the length of the catheter assembly until the point 258 at which the tapered geometry segment 218 begins to taper, at which point only the first lumen 206, which provides the fluid path through the catheter assembly 200, continues in the distal direction. As shown in FIG. 10B, the catheter assembly 200 may further include a depth stop 232, as described elsewhere herein.

With reference to FIGS. 11A-11B, another dual lumen (e.g. first lumen 206 and second lumen 252) embodiment of a catheter assembly 200 is illustrated. In contrast with the embodiment shown in FIGS. 10A-10B, the catheter assembly 200 of FIGS. 11A-11B includes a shoulder 260 disposed about the closed, distal end 256 of the second lumen 252, rather than simply terminating the second lumen 252 at the point 258 at which the tapered geometry segment 218 begins. The shoulder 260 may form a blunt depth stop 232 that limits an extent to which the distal tip 214 of the catheter assembly 200 is insertable in use through the RWM. In certain embodiments, as shown in FIG. 11B, the catheter assembly 200 includes a tapered geometry segment 218 in addition to the blunt shoulder 260. In this embodiment, the tapered geometry segment 218 may be disposed about the first lumen 206 only, and may be partially or entirely disposed at an axial position that is distal of the shoulder 260. In other embodiments, as shown in FIG. 11A, the distal tip 214 may be blunt, and the tapered geometry segment 218 may be omitted. In such embodiments, the cross-sectional dimensions of the catheter assembly 200 may remain substantially consistent along the axial extent of the portion of the catheter assembly 200 that extends distally beyond the shoulder 260.

Turning next to FIGS. 12A-12B, in further embodiments, the catheter assembly 200 may include a rigid segment 262 disposed within the overcoating or jacket 202, with an integrated needle 264 at the distal tip 214 thereof. The needle 264 may be disposed, e.g., on the distal end or tip of the rigid segment 262, such that the needle 264 extends distally from a distal end thereof. The rigid segment 262 may be made of, e.g., metal. The tip of the needle 264 may include any known geometry, and the lumen of the needle 264 may be in fluid communication with a liner 204 disposed within the metal segment 262 (FIG. 12B). Such a catheter assembly may be used for single-step introduction into the RWM and infusion. A depth stop 232 such as, e.g., an integrated depth stop or a depth stop 232 coupled to the radially outward facing surface of the catheter assembly 200 may be provided to prevent over-insertion.

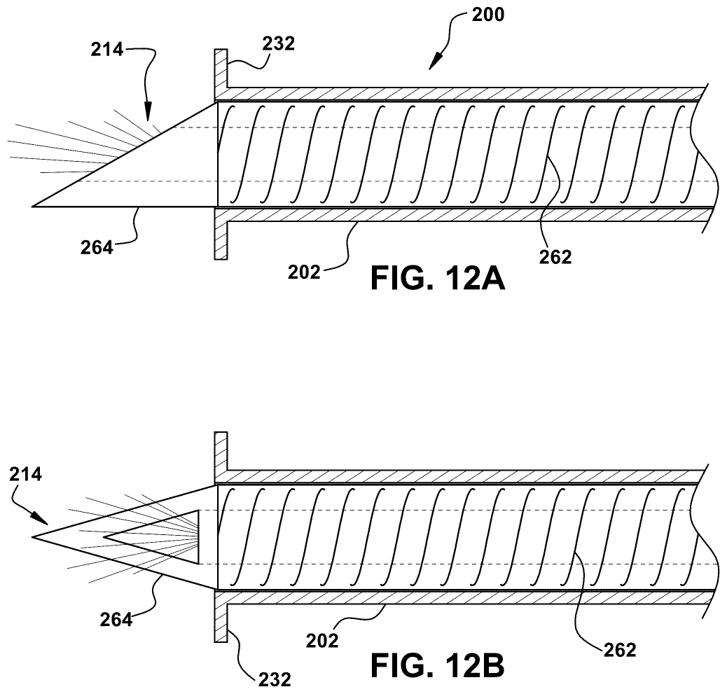

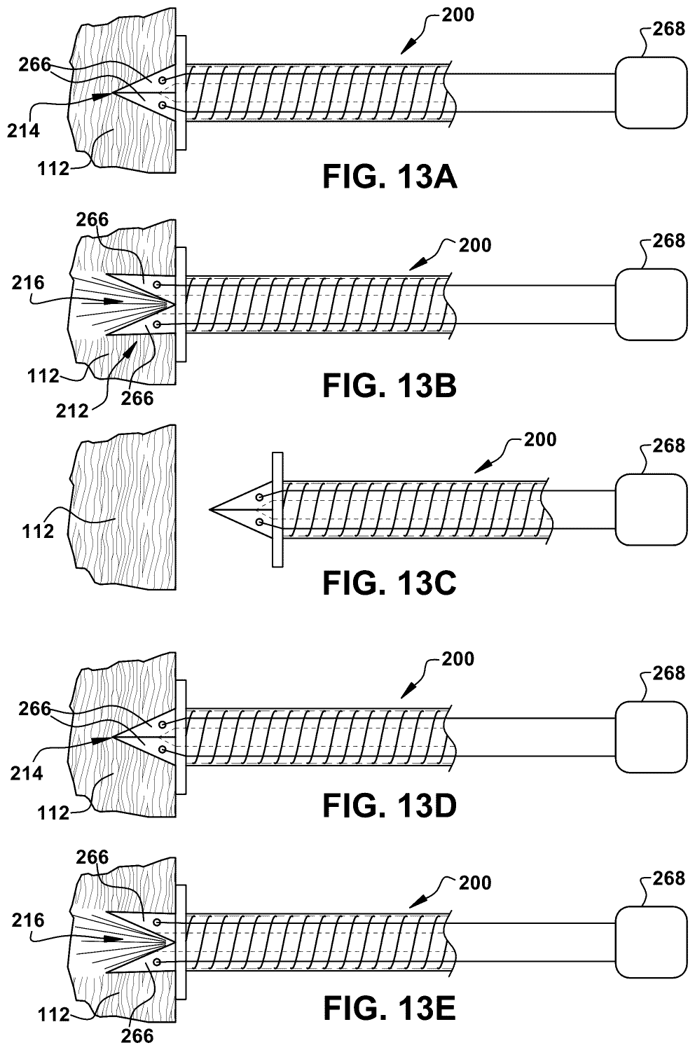

Turning to FIGS. 13A-13E, in certain embodiments, the catheter assembly 200 may include a pair of tip members 266 that collectively form the distal tip 214. Each tip member 266 of the pair of tip members 266 is movable between a closed position (FIGS. 13A, 13C, 13D) and an open position (FIGS. 13B, 13E). In the closed position, the pair of tip members 266 collectively form a sharp point at a distal end 214 thereof, which may be adapted to puncture the RWM 112. In the closed position, there is no opening (contrast with the presence of opening 216 in, e.g., FIG. 7A) through which drug product may flow from the catheter assembly 200. To transition to the open position, the pair of tip members 266 are adapted to articulate radially outwardly to reveal an opening 216 at the distal end 212 of the catheter assembly 200. A drug product may be infused into the RWM through the opening 216. Following the infusion, the pair of tip members 266 may be returned to the closed position for withdrawal from the subject. A microcontroller 268 may be provided, to selectively move the pair of tip members 266 between the closed position and the open position. In various embodiments, the microcontroller may include, e.g., a micromanipulator. The micromanipulator may encompass any instrument adapted to allow physical interaction with a small scale substrate such as, e.g., a sample viewable on a microscope stage, a microelectronic device, or an in vivo experiment or procedure. Such interaction may take place with or without the aid of a microscope. The micromanipulator may provide a level of precision in movement and positioning that is unattainable with an unaided human hand. In certain embodiments, the micromanipulator may include an input controller such as, e.g., a joystick, a mechanism for reducing the range of movement including, e.g., kinematic constraints, and an output unit adapted to hold and control a microtool capable of manipulating an object such as, e.g., the catheter assembly 200 including tip members 266. The micromanipulator may further be adapted to operate hydraulically or pneumatically to open and close the tip members 266 catheter. The micromanipulator may further be motor-driven in certain embodiments. Exemplary micromanipulators are commercially available and will be understood by those skilled in the art.

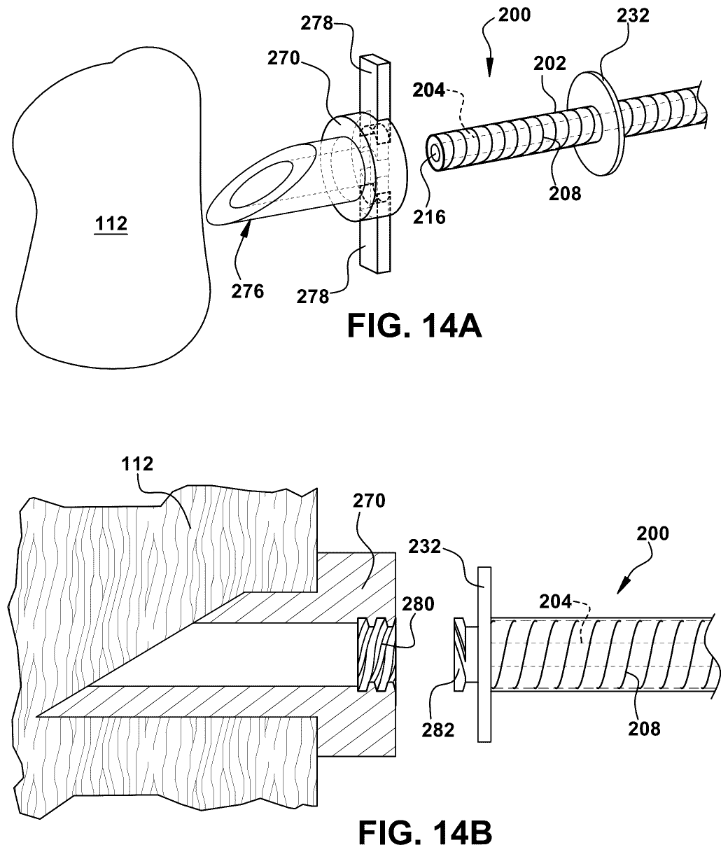

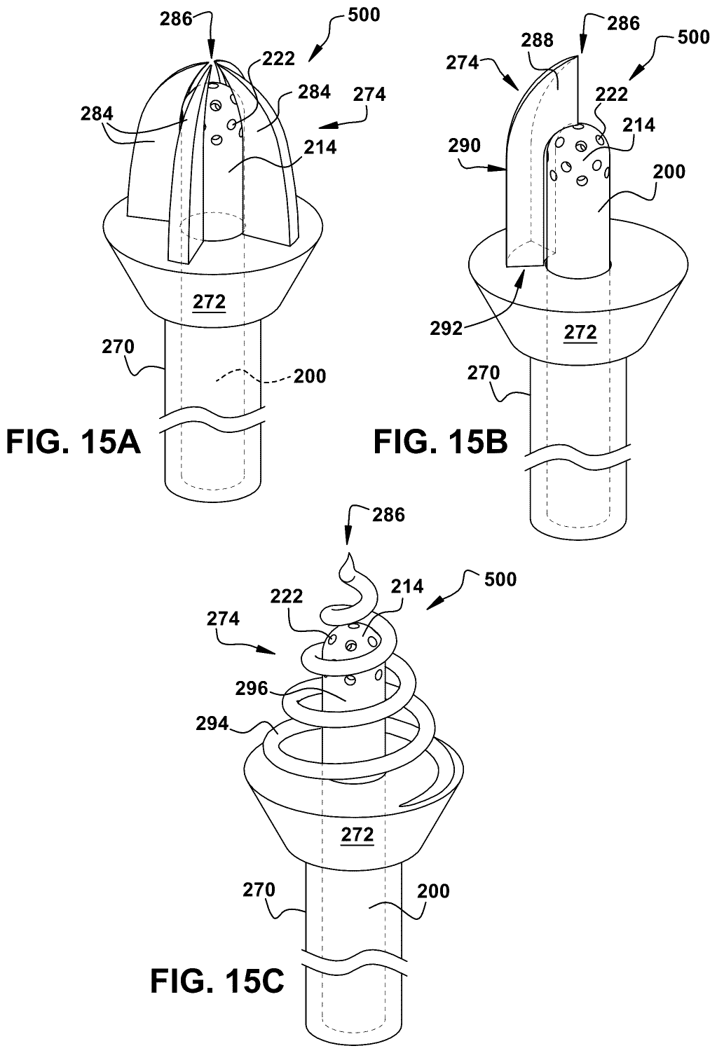

According to certain embodiments, such as those shown in FIGS. 14A-14B and 15A-15C, the catheter assembly 200 may further include an introducer 270 disposed over at least a portion, e.g., a distal portion, of the catheter assembly 200. Referring to the embodiment of FIG. 14A, in certain embodiments, the introducer 270 may be in the form of a hub, and may include a support structure at a distal end thereof. The support structure may be, e.g., a needle 276 extending distally therefrom. The hub may be, e.g., a molded hub. The introducer 270 may include a plurality of engagement features such as, e.g., pinch blocks 278 adapted to engage a portion of the catheter assembly 200. The pinch blocks 278 may be finger-operated, and adapted to engage a depth stop 232 disposed on the catheter assembly 200 when pressed by a user. This engagement between the pinch blocks 278 and the catheter assembly 200 functions to fix and maintain an axial position of the catheter assembly 200 relative to the introducer 270. FIG. 14A illustrates the use of two pinch blocks 278, although other embodiments including three, four, or more pinch blocks 278 are also contemplated. The catheter assembly 200 may include a blunt or substantially blunt distal end terminating in an opening 216 (as in, e.g., FIG. 5B), which may be in fluid communication with the needle 276 of the introducer 270 when the catheter assembly 200 is inserted into the introducer 270.

In FIG. 14B, another embodiment is shown which includes an introducer 270 in the form of a hub. The introducer hub may include a needle as in FIG. 14B, or another support structure. In the embodiment of FIG. 14B, the introducer 270 includes at a proximal end thereof, an inlay 280 having a connector, e.g., a luer lock. A distal end of the catheter assembly 200 may be inserted in a distal direction into the introducer 270 via the inlay 280. The catheter assembly 200 may include a depth stop 232 having a connector 282 disposed thereon. The connector 282 may be adapted to matingly engage the connector in the inlay 280, such that the connector on the inlay 280 and the connector 282 on the catheter assembly 200 may be, e.g., complementary luer lock connectors, which facilitates the mating engagement between the introducer 270 and the catheter assembly 200. In this manner, the depth stop 232 on the catheter assembly 200 may interact with the introducer 270 to limit the freedom of the catheter assembly 200 to translate distally, preventing over-insertion of the catheter assembly 200 relative to the introducer 270. The position of the depth stop 232 having connector 282 thereon, along the axial extent of the catheter assembly 200 may be selected based on a number of factors including, e.g., the length of the introducer 270, and the pre-determined extent, if any, that the catheter assembly 200 will extend beyond distal end of the introducer 270.

In use, the introducer 270 of FIGS. 14A-14B may be inserted into the RWM 112, and the catheter assembly 200 may be engaged in the introducer 270 and inserted into the cochlea via the introducer 270. Alternatively, the catheter assembly 200 may be inserted into the introducer 270 and engaged therein prior to insertion into the RWM 112. In any case, upon completion of the administration of the drug product, the introducer 270 and catheter assembly 200 may be withdrawn as a single unit from the subject.

Turning next to FIGS. 15A-15C, further embodiments of catheter assemblies 200 including an introducer 270 are shown, in which the introducer 270 is in the form of a sheath. In the embodiment of FIG. 15A, the introducer 270 includes a depth stop 272, and a support structure 274 including a plurality of blades or prongs 284 extending distally from the depth stop 272 disposed near the distal end of the introducer 270. The blades or prongs 284 may be circumferentially arranged and spaced about the distal tip of the sheath 270. In certain embodiments, the blades or prongs 284 may be evenly spaced about the circumference of the depth stop 272. Four such blades or prongs 284 are illustrated in FIG. 15A, although any number of blades or prongs may be used, e.g., two, three, four, or more blades or prongs 284. The blades or prongs 284 may have an outer surface that is arcuate, curved, or tapered as the blades or prongs 284 extend distally, to collectively form a sharp distal tip 286. The sharp distal tip 286 may be adapted to puncture the RWM in use. The catheter assembly 200 may be disposed within the introducer 270, and axially positioned within the introducer 270 such that the distal tip 214 of the catheter assembly 200 is disposed within, and shielded by the blades or prongs 284. The distal tip 214 of the catheter assembly 200 may include a plurality of through-holes 222 extending from the overcoating or jacket 202 of the catheter assembly 200 to the lumen therein containing the drug product. In use, the drug product may be infused via the through-holes 222. Spaces between the axially-extending blades or prongs 284 allow the infused drug product to move beyond the support structure 274 and infuse into the RWM of the subject.

FIGS. 15B and 15C illustrate additional exemplary embodiments of catheter assemblies 200 including an introducer 270 in the form of a sheath having a support structure 274. As shown in FIG. 15B, the support structure 274 includes a sconce 288 extending distally from the depth stop 272, and disposed about a portion of a circumferential extent of the distal tip 214 of the catheter 200. The sconce 288 may extend about a partial extent of the circumference of the introducer 270. For example, the sconce 288 may extend about 90°, about 100°, about 110°, about 120°, about 130°, about 140°, about 150°, about 160°, about 170°, about 180°, about 190°, about 200°, about 210°, about 220°, about 230°, about 240°, about 250°, about 260°, or about 270° about the circumference of the introducer 270, thereby partially enclosing the distal tip 214 of the catheter assembly 200. The sconce 288 may have an outer surface 290 that is arcuate, curved, or tapered along its axial extent in the distal direction, to terminate at a sharp distal tip 286, which may be adapted to puncture the RWM in use. The sconce 288 may also include an axially extending opening 292, which spans the remainder of the extent of the circumference of the introducer 270, i.e., the extent of the circumference over which the sconce 288 does not extend. For example, where the sconce 288 extends about 180° around the circumference of the introducer 270, the opening 292 may extend a complementary extent of about 180° around the introducer 270. In another example, where the sconce 288 extends about 200° about the circumference of the introducer 270, the opening 292 may extend about 160°. In certain embodiments, the sconce 288 may extend 180° or more around the distal tip 214 of the catheter assembly 200.

In FIG. 15C, the support structure 274 includes a helical structure 294 circumferentially disposed about the distal tip 214 of the catheter 200. The curve of the helical structure 294 extends distally from the introducer 270, or more particularly, from a depth stop 272 disposed near the distal end of the introducer 270. The helical structure 294 may be, e.g., a conical helix, in which the base of the conical volume is aligned or substantially aligned with the distal end of the introducer 270, and the height of the conical volume extends distally to a vertex that forms the distal tip 286, which may be adapted to puncture the RWM. The helical structure 294 may be right or left-handed, and may include openings 296 between turns of the helical structure 294. In other embodiments, the helical structure 294 may include a cylindrical helix which transitions to a conical helix at a distal end thereof.

As in FIG. 15A, the catheter assemblies 200 of FIGS. 15B and 15C may be disposed within the introducer 270, and axially positioned within the introducer 270 such that the distal tip 214 of the catheter assembly 200 is disposed within, and shielded by the support structure 274, e.g., sconce 288 or helical structure 294. The distal tip 214 of the catheter assembly may include a plurality of through-holes 222 extending from the outer surface of the catheter assembly 200 to the lumen therein containing the drug product as shown in, e.g., FIG. 5A. In use, the drug product may be infused into the cochlea of the subject via the through-holes 222. The spaces between the blades or prongs 284 (FIG. 15A), the openings 292 (FIG. 15B) or the openings 296 (FIG. 15C) as applicable, allows the infused drug product to move beyond the support structure 274 into the cochlea via the RWM of the subject.

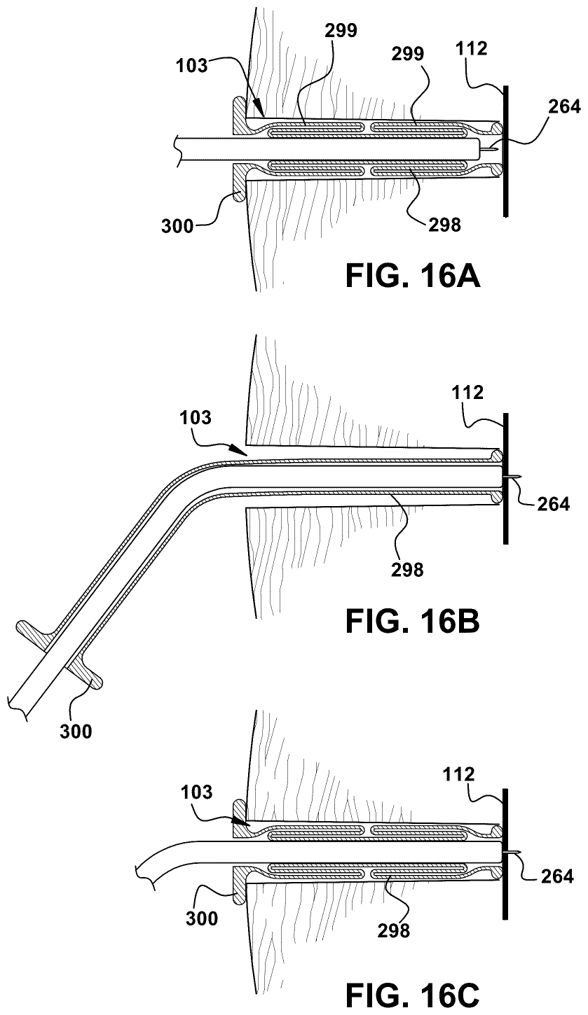

Turning next to FIGS. 16A-16C, embodiments are provided herein in which a catheter assembly 200 includes a needle such as, e.g., needle 264. A housing 298 may be disposed over the catheter assembly 200. The housing 298 may be flexible and collapsable, and may be movable between a flexible, extended state (FIG. 16B) and a rigid, collapsed state (FIG. 16C). The housing 298 is configured to at least partially, e.g., substantially contain the otic catheter assembly 200 while allowing the needle, e.g. needle 264, to extend distally beyond a distal end of the housing 298.

As best seen in FIG. 16A, in certain embodiments, the housing 298 may comprise a plurality of concentrically arranged components 299 which are adapted to translate distally or proximally with respect to one another in order to expand or contract the housing 298, respectively. For example, one or more concentric components 299 may translate away from another concentric component 299 to expand the length of the housing 298. In this state, shown in FIG. 16B, the housing 298 may be comparatively flexible, and movement around a central axis of the housing 298 may be permitted. In this state, the housing 298 may also be permitted to bend. The concentric components 299 may also be adapted to translate toward one another to arrive at a collapsed state (FIG. 16C). In this collapsed state, the housing 298 may be substantially more rigid, and may have a shorter axial length as compared to the expanded, flexible state. In the collapsed state, movement about the central axis of the housing 298 and bending of the housing 298 are resisted by the stiffness of the housing 298 associated with the close fit of the concentric components. In the rigid, collapsed state, the housing 298 is configured to secure and maintain a position and an orientation of the needle 264.

The housing 298 may further include a flange 300 disposed at a proximal end thereof. As best shown in FIG. 16C, the flange 300 may have an outer diameter selected to be larger than a diameter of the access hole 103 drilled in the subject's skull to facilitate the trans-mastoid surgical approach (see FIG. 1). In this manner, the flange 300 is configured to act as a depth stop, limiting the extent of insertion of the housing 298 and catheter assembly 200 disposed therein, through the access hole 103 in the subject's skull. The depth to which the needle 264 may be inserted through the RWM 112 may be predetermined. Without limitation, the depth may be determined based on one or more attributes or dimensions of the housing 298 and the catheter assembly 200, such as an interior profile of the housing 298, a depth to which the catheter assembly 200 may be inserted into the housing 298, and a depth to which the housing 298 may be inserted through the access hole 103 into the skull. The length of the housing 298 from the flange 300 to the distal tip, the interior profile of the housing 298, and the length of the needle 264 may all be pre-selected in order to achieve a desired depth of insertion.

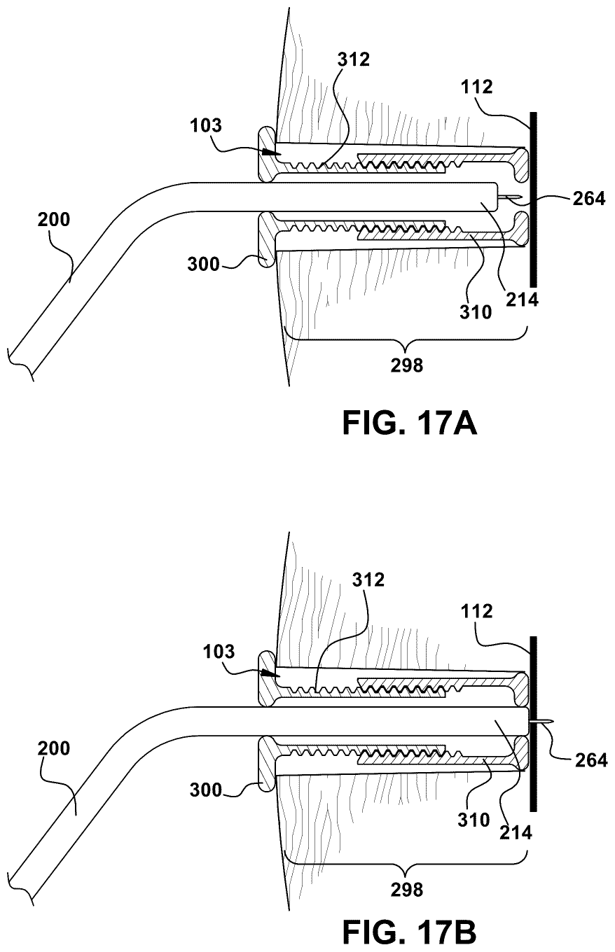

With reference to FIGS. 17A-17B, another catheter assembly 200 having a needle such as, e.g., needle 264, and a housing 298 disposed thereover is shown. According to the embodiment of FIGS. 17A-17B, the housing 298 may include a distal housing member 310 that is disposed over the distal tip 214 of the otic catheter assembly 200, and configured to limit distal movement thereof upon contact by the distal housing member with the RWM 112. As noted, the catheter assembly 200 may include a needle 264, which may be permitted to extend distally beyond the distal housing member 310. A proximal housing member 312 is coupled to the distal housing member 310. Collectively, the distal and proximal housing members 310, 312 form the housing 298. A flange 300 may be provided at a proximal end of the housing 298, particularly on the proximal end of the proximal housing member 312. The flange 300 may be coupled to the proximal housing member 312, disposed over the proximal housing member 312, or may be integrally formed therewith. The flange 300 may be configured to engage an outer surface of a skull of the subject at a periphery of the access hole 103. As used herein, “skull” is understood to refer to the bony structure of the subject's head, e.g., all bone and tissue thereof, and including all surfaces thereof, e.g., an outer surface as well as an inner surface of any bores or holes created therein. Through engagement of the flange 300 with the skull, distal movement of the housing 298 is limited relative to the skull.

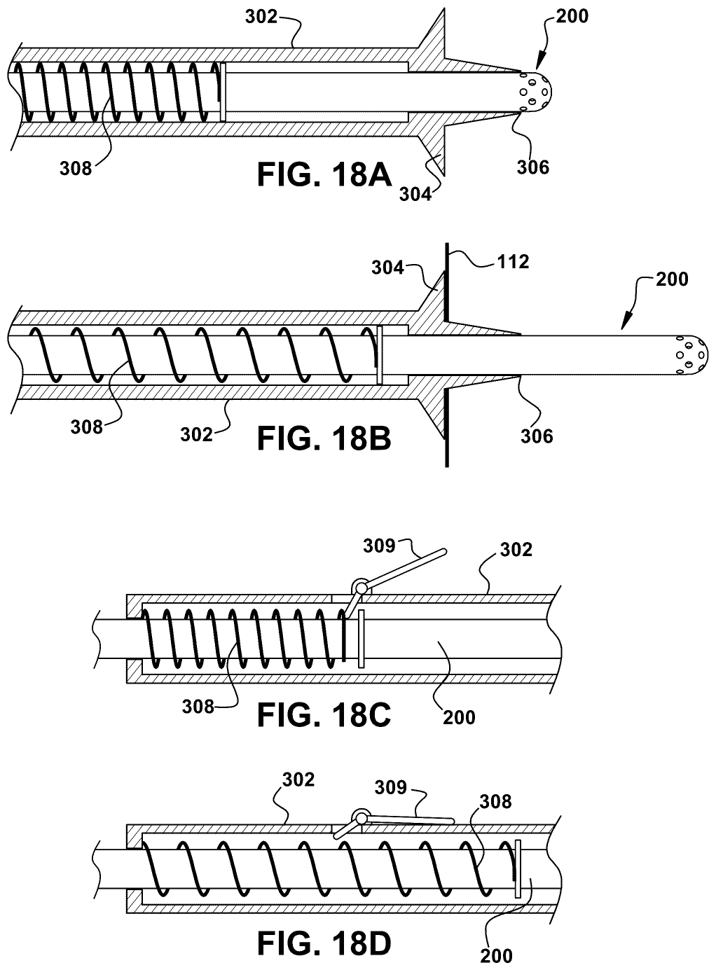

Turning next to FIGS. 18A-18D, an embodiment is illustrated in which an otic catheter assembly 200 is provided within the lumen of an introducer 302, which may be in the form of a needle. The introducer 302 may include a depth stop 304 disposed thereon, e.g., on a radially outward facing surface of the introducer 302. The depth stop 304 may be configured to limit the extent of insertion of the needle into the subject and through the RWM, and to anchor the introducer 302 to the exterior of the RWM. The introducer 302 may further include a sharp distal tip 306 disposed distally relative to the depth stop 304, and adapted to pierce the RWM during use.

A spring or other biasing member 308 may be disposed within a lumen of the introducer 302, and may be adapted to bias the otic catheter assembly 200 in a distal direction. The catheter assembly 200 may be, e.g., a microcatheter. As shown in FIG. 18C and FIG. 18D, upon full bevel insertion of the sharp distal tip 306 of the introducer 302, the user may press an actuator 309, e.g., a spring release button, to release the biasing element 308. This in turn biases the catheter assembly 200 in the distal direction. Despite the biasing force, the catheter assembly 200 may not immediately deploy in the distal direction due to resistance from the RWM. The user may then continue to advance the catheter assembly 200 through the RWM until the catheter assembly 200 (e.g., microcatheter) deploys, indicating that the other side of the RWM has been reached. The depth stop 304 on the exterior of the RWM may act as a depth limiter, preventing or limiting the extent of insertion. In certain embodiments, the position of the depth stop 304 may be adjustable to account for anatomical differences between patients.

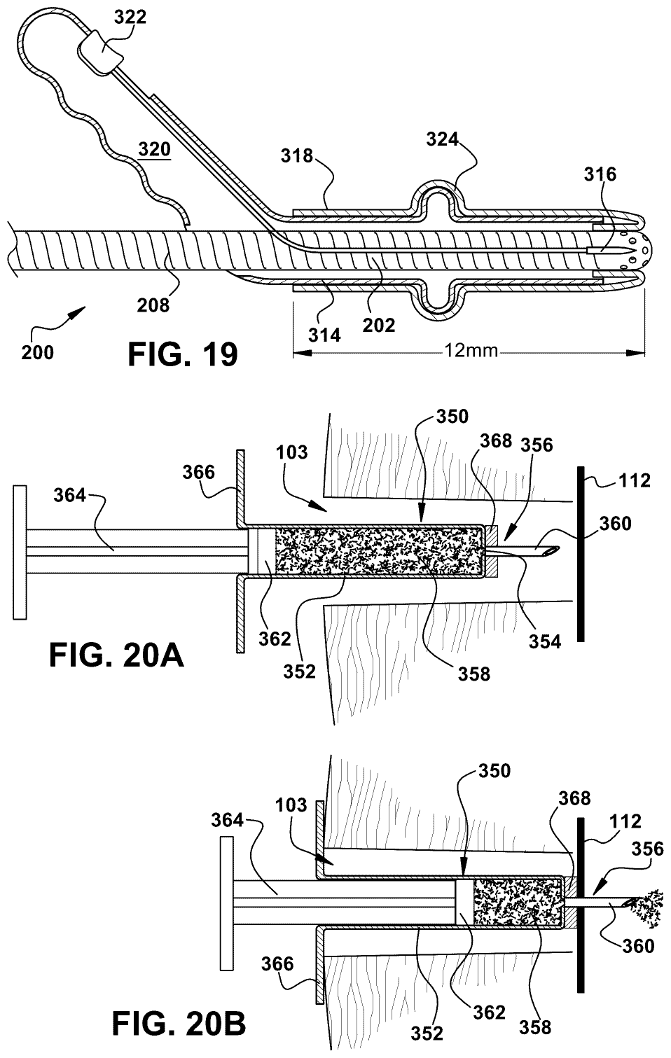

With reference to FIG. 19, a further embodiment is illustrated, in which the catheter assembly 200 includes an integrated and actuatable pick adapted to puncture the RWM. The catheter assembly 200 includes a first sleeve 314 disposed over the outermost layer of the catheter assembly 200, e.g., over the overcoating or jacket 202. A pick 316 is disposed within a lumen in the first sleeve 314, and may be oriented substantially parallel to the catheter assembly 200. Upon actuation, the pick 316 is configured to translate distally to puncture the RWM, in an action analogous to a “serpent's tongue.” A handle 320 is coupled to a proximal end of the first sleeve 314, and includes an actuator 322 for actuating translation of the pick 316. The handle 320 may further include an ergonomic grip for the case of the user. A second sleeve 318, which may be, e.g., an over-molded rigid sleeve, is disposed over at least a portion of the first sleeve 314. The second sleeve 318 may further include a depth stop 324 disposed thereon and configured to limit a depth to which the otic catheter assembly 200 is insertable into a patient. For example, the distance from the distal tip of the second sleeve 318 to the distal face of the depth stop 324 may be, e.g., about 12 mm.

Turning to FIGS. 20A-20B, a terminally sterilized, pre-filled syringe 350 is provided for use in administering an otic injection. The syringe 350 includes a barrel 352 having an outlet 354 at a distal outlet end 356, and a flange 366 disposed on the proximal end of the barrel 352 opposite the outlet end 356. The syringe barrel 352 contains a drug product 358 for otic, e.g., intracochlear administration. A needle 360 is disposed at the outlet end 356, and may be in fluid communication with the outlet 354. A stopper 362 is disposed within the barrel and axially translatable therein, as actuated by a plunger rod 364. In use, the plunger rod 364 contacts the stopper 362 and biases the stopper 362 is the distal direction, i.e. toward the outlet end 364 of the barrel 352. A spacer 368 is disposed about the needle 360 at the outlet end 356.

In use, the syringe 350 may be inserted through the subject's skull via the access hole 103. The flange 366 at the proximal end of the barrel 352 may be used to anchor the position of the syringe 350 relative to the skull. In certain embodiments, the flange 366 may also serve as a depth stop to limit an extent of insertion of the syringe 350. The spacer 368 may be configured to abut the RWM with a distal face thereof, and to abut the outlet 354 with a proximal face thereof. This serves to limit a depth to which the needle 360 may be inserted in use, as the spacer 368 limits the portion of the length of the needle 360 that may be inserted into the RWM to only that portion extending distally beyond the spacer 368. The thickness of the spacer 368 may be predetermined based on, e.g., a length of the needle 360 that extends beyond the outlet 354 of the syringe 350, and a desired depth of insertion of the needle 360 into the RWM. Other factors may also be considered in predetermining the thickness of the spacer 368 such as, e.g., a length of the barrel 352 from the flange 366 to the outlet 354, and other dimensions. The length of the barrel 352 from the flange 366 to the outlet 354 in particular may be pre-selected to allow the needle 360 to reach the RWM via the access hole 103. In one exemplary embodiment, the syringe 350 may be, e.g., a 0.5 mL syringe having an 8 mm needle, and a 15 mm stroke distance may deliver about 0.24 mL of drug product to the cochlea of the subject. Other syringe lengths, volumes, dosages, and stroke distances are also contemplated as part of the disclosure.

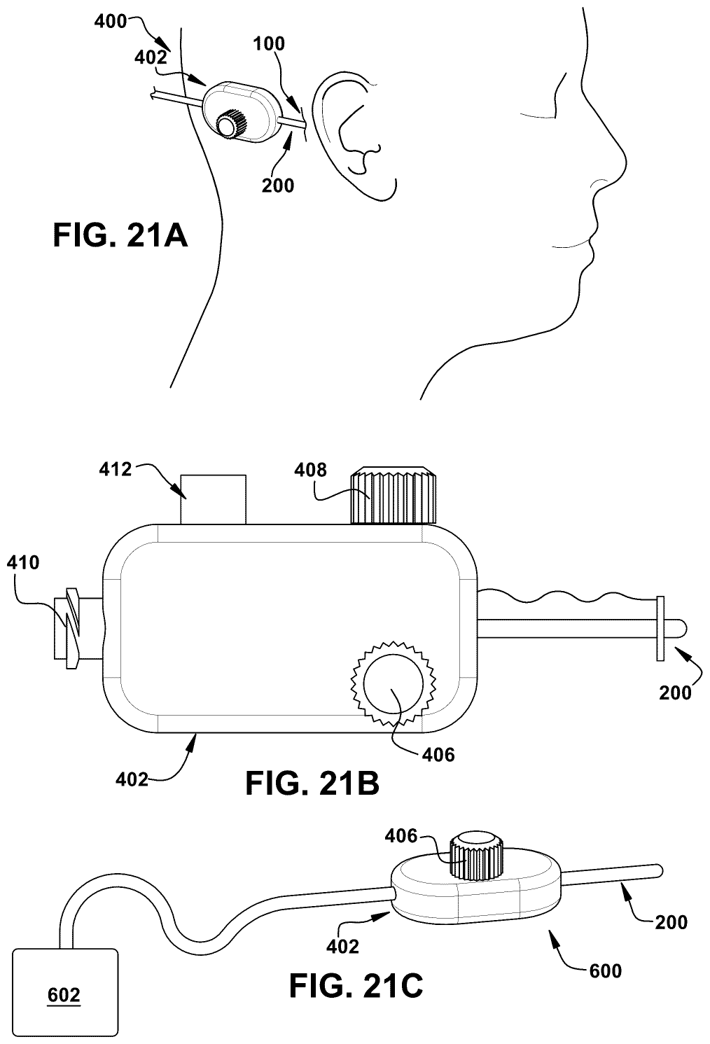

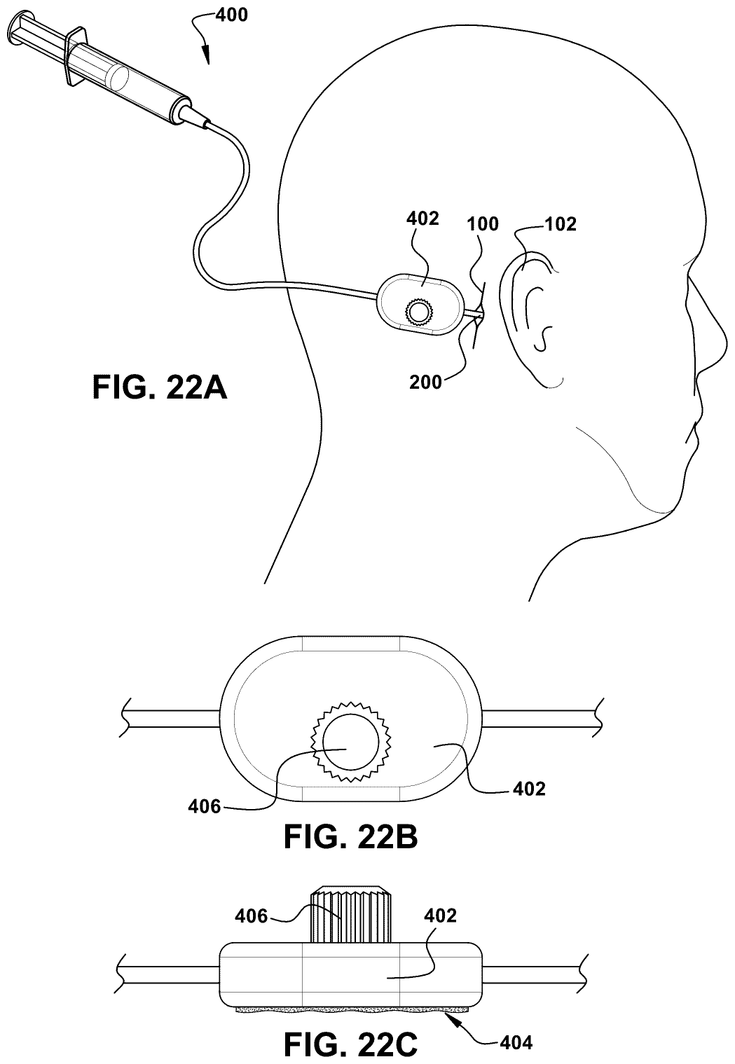

Turning to the embodiments of FIGS. 21A-21C and 22A-22C, otic infusion systems 400 are disclosed, including an otic catheter assembly 200 as described elsewhere herein. For example, the otic catheter assembly 200 may include a liner or sheath 204 having a first lumen 206 axially extending therethrough; an overcoating 202 optionally disposed over the liner 204; and a distal tip 214 of the catheter assembly 200 that is adapted for otic administration of a drug product, e.g., for intracochlear insertion through the round window membrane (RWM). The system 400 may further include a feed device 402 disposed on a patient's skin at a location proximate to an incision 100 in the skin through which the RWM 112 (FIG. 1) is accessible. The feed device 402 is configured to receive the otic catheter assembly 200 therethrough, and may include an adhesive 404 (FIG. 22C) configured to adhere to the patient's skin to securely maintain a position of the feed device 402 during use (FIGS. 21A and 22A).

As shown in, e.g., FIGS. 21B-21C and 22B-22C, the feed device 402 may include a rotatable dial or knob 406 configured to advance the otic catheter assembly 200 into the RWM, and to retract the otic catheter assembly 200 from the RWM. For example, the dial or knob 406 may be rotated in a first direction, e.g., clockwise, to advance the catheter assembly 200, and in a second, opposite direction, e.g., counter clockwise, to retract the catheter assembly 200. In certain embodiments, the rotatable dial or knob 406 includes a micro-adjustment knob, which allows for very small and incremental adjustments and advancements of the catheter assembly 200, to minimize, limit, or avoid damage to the internal car structures (FIG. 3). As shown in FIG. 21B, in certain embodiments, a first dial or knob 406 may be provided for adjusting the position of the catheter assembly 200 along the X axis, and a second dial or knob 408 may be provided for adjusting the position of the catheter assembly 200 along the Y axis.

In either case, the catheter assembly 200 may be inserted into the feed device 402 via an opening 412, and may translate through the feed device 402, as actuated by knob or dial 406 (and 408 if present) during insertion into the RWM of the subject. The delivery device, e.g., catheter assembly 200, is maintained in a taut condition during insertion to prevent any kinks from forming therein, which may impact the flow of the infusion.

The feed device 402 may further include a connector 410, e.g., a luer connection, which may be at a proximal end thereof. The connector 410 may be configured to fluidly couple the otic catheter assembly 200 to a device such as, e.g., an infusion pump which is adapted to administer the infusion.

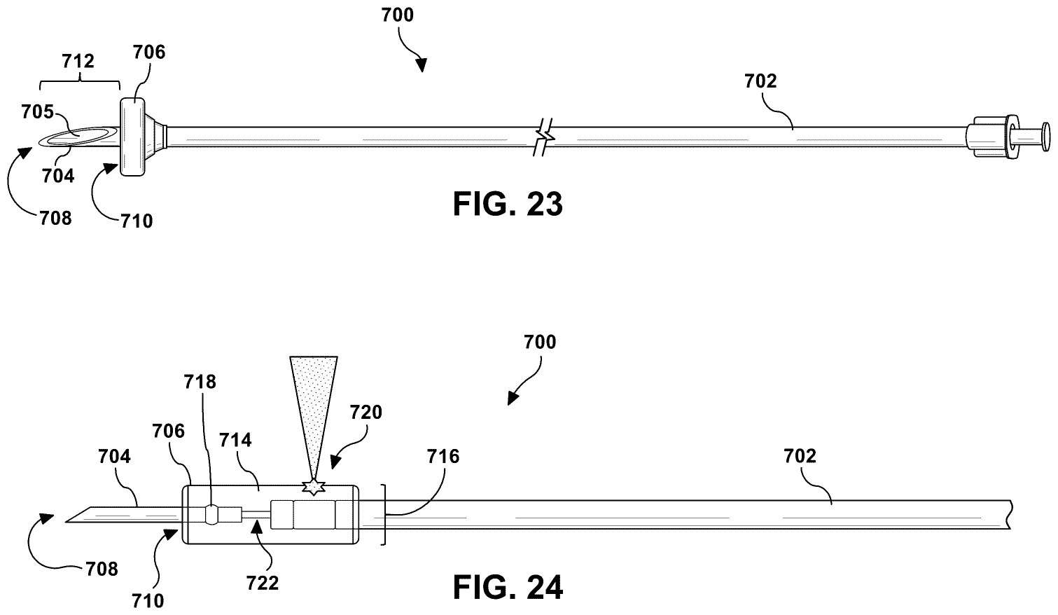

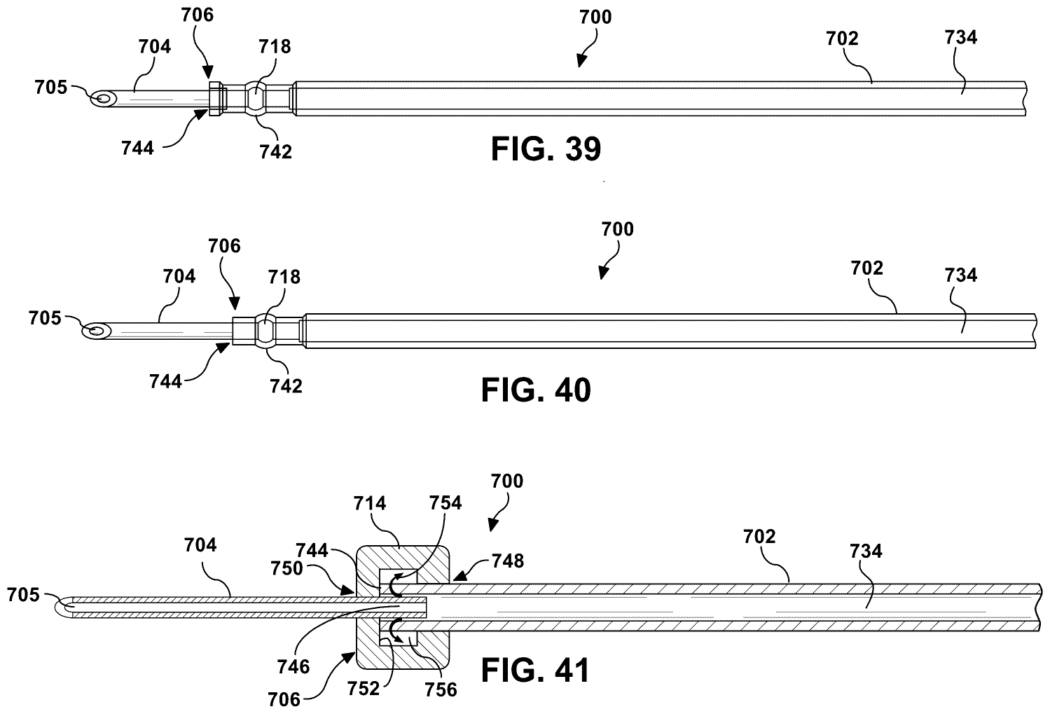

Turning to FIGS. 23-53, additional embodiments of otic catheter assemblies 700, and components thereof, are shown. In certain embodiments, the otic catheter assembly 700 includes a catheter 702 and an integrated microneedle 704. Various embodiments may also include a depth stop 706. The microneedle 704 may be shaped and dimensioned to facilitate insertion of the tip of the microneedle 704 into the RWM 112 to a depth at which the depth stop 706 contacts the RWM 112, in a manner similar to that shown in, e.g., FIG. 9B, wherein the distal tip 214 of the otic catheter assembly 200 is inserted into the RWM 112 until the depth stop 232 contacts the RWM 112. The depth stop 706 may be disposed proximally relative to the distal tip 708 of the microneedle 704. The depth stop 706 may further extend radially outward from the outer surface of the catheter assembly 700. In certain examples, the depth stop 706 may extend 360° around the circumference of the catheter assembly 700, while in other examples, the depth stop 706 may extend only partially, or intermittently about the circumference of the catheter assembly 700, as discussed further herein below. The depth stop 706 may be integrally formed with the catheter 702 (FIGS. 39-40), or may be a feature of a separately fabricated component that is coupled thereto, e.g., the depth stop 706 may be a feature of a hub 714 (e.g., FIGS. 24-38).

The distal tip 708 of the microneedle 704 may be adapted for insertion through the RWM 112, and the axial distance 712 (FIG. 23) from the distal tip 708 of the microneedle 704 to a distal face 710 of the depth stop 706 may correspond to the desired depth to which the catheter assembly 700 is to be inserted into the RWM 112 in use. For example, the desired depth may be, e.g., about 5 mm or less, about 4 mm or less, about 3 mm or less, about 2 mm or less, or about 1 mm or less. At the distal tip 708, the needle may include a bevel 730 (see FIG. 44), which may have a length 762 that is relatively short, a result of a primary bevel angle that is relatively high. For example, conventional needles may have a primary bevel angle of about 19°, to provide a bevel with sufficient length to maintain sharpness of the tip. However, the bevel 730 may have a primary bevel angle of, e.g., 45° or less, 40° or less, 35° or less, 30° or less, or 25° or less. Such bevel angles are considered relatively high, and result in a shorter bevel tip than that of a needle having a lower, more conventional bevel angle of, e.g., 19°. The use of a relatively short bevel, having a relatively high bevel angle minimizes the distance the needle is inserted beyond the RWM during use, facilitating delivery of the drug product with minimal opportunity for contact between the needle and delicate anatomical structures of the cochlea. A relatively sharp distal tip 708 is also desirable in order to puncture the tough and fibrous RWM. The microneedle 704 may be a small, conventional gauge needle such as, e.g., about 28 gauge or smaller. For example, the microneedle 704 may be a 28 gauge needle, 29 gauge, 30 gauge, 31 gauge, 32-gauge, and so on. Alternatively, the microneedle 704 may be a silicon needle.

The catheter 702 and microneedle 704 may be integrated according to any of a number of embodiments disclosed herein. Further, the depth stop 706 may adopt any of a number of shapes and configurations, including but not limited to those described herein. Further, various features and embodiments depicted herein may be combined with various other features and embodiments described herein, as will be appreciated by one of skill in the art.

Various otic catheter assemblies 700 disclosed herein include a hub 714 to facilitate integration of the microneedle 704 with the catheter 702, as shown in, e.g., FIGS. 24-26, 29, 31, 33, 35-38, and other figures. Various components, e.g., the hub 714 and the catheter 702, may be affixed to one another using welding techniques such as, e.g., socket laser weld.

As shown in, e.g., FIGS. 24-26, in certain embodiments, the proximal end of the microneedle 704, opposite the distal tip 708, may be fluidly coupled with the distal end of the catheter 702, within a hub 714. The hub 714 may be made of, e.g., plastic, and may in some embodiments be transparent or translucent, allowing clear to partially clear visualization of the proximal end of the microneedle 704 and the distal end of the catheter 702. The hub 714 includes a longitudinally extending lumen 722 disposed therethrough, extending from the distal end of the hub 714 to the proximal end thereof.

The outer diameter 716 of the hub 714 at the distal end thereof may be selected to provide a depth stop 706 adapted to contact the RWM 112 at the distal face 710 of the hub 714. Contact between the distal face 710 and the RWM 112 may provide tactile feedback to the user upon insertion of the microneedle 704 through the RWM 112 to the desired depth.

The hub 714 may be insert-molded with the microneedle 704. The microneedle 704 may include a retention feature such as, e.g., a formed bead 718 (e.g., FIGS. 24-26, and others), which may be disposed at a point along the shaft of the microneedle at or near the proximal end thereof. The retention feature, e.g., bead 718, may contribute to the mechanical interlock between the microneedle 704 and the hub 714, such that axial translation of the microneedle 704 is limited, e.g., prevented relative to the hub 714. The bead 718 may be, e.g., a portion of the microneedle 704 having an increased outer diameter relative to the outer diameter of the shaft of the microneedle 704. The lumen 705 (best shown in, e.g., FIGS. 23, 33, 35, 36, 39-41, 44-47) in the microneedle 704 may be in fluid communication with the lumen 722 of the hub 714. The size or gauge of the microneedle 704 may be selected to provide minimally invasive access to the cochlea through the RWM 112, and may be, e.g., about 32-gauge in certain embodiments.

As shown in FIGS. 24-26, 29, 31, 33, and others, the distal end of the catheter 702 may be received within the proximal end of the hub 714, such that the lumen 734 in the catheter 702 is in fluid communication with the lumen 722 of the hub 714 and the lumen 705 within the microneedle 704. In certain embodiments, the catheter 702 may be made of an opaque material, facilitating visibility within a hub 714 that may be made of transparent or translucent material.

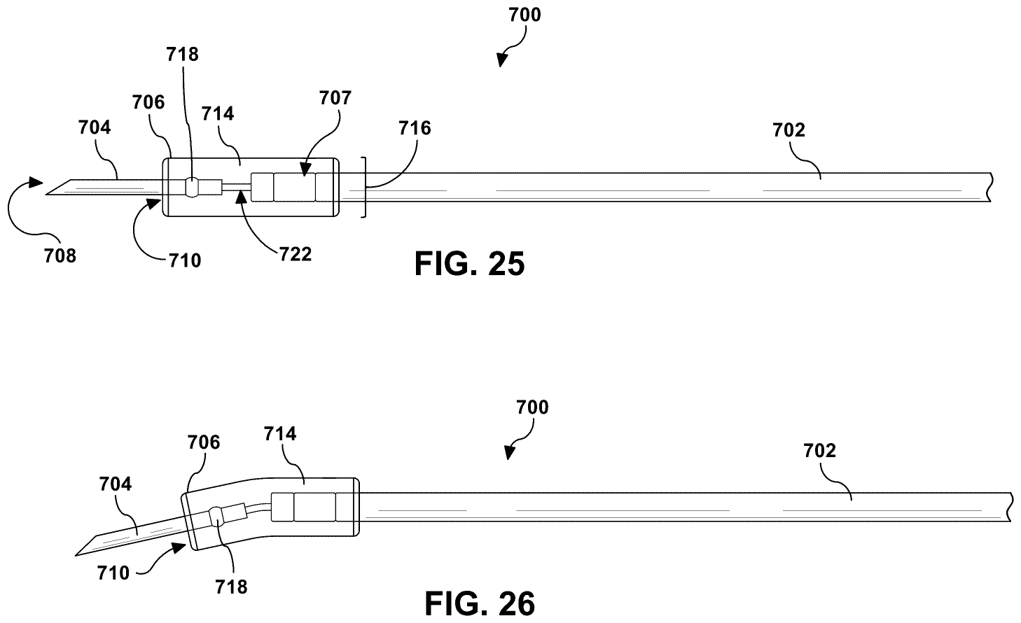

In certain embodiments, such as shown in FIG. 24, a radial laser weld 720 may be used to secure the distal end of the catheter 702 within the hub 714. In other embodiments, such as shown in FIG. 25, an adhesive or glue 707 may be used. For example, a glue 707 such as, e.g., a wicking, UV-cured epoxy (such as those sold under the LOCTITE brand) may be applied to the joint, e.g., between the outer surface of the catheter 702 and the hub 714.

In certain embodiments, as shown in, e.g., FIGS. 24-26, the hub 714 may be substantially annular in cross-sectional shape, and may have an outer diameter 716 (FIG. 24) that is consistent or approximately consistent along the length of the hub 714. The outer diameter 716 of the hub 714 may be greater than the outer diameter of the needle 704 and also greater than the outer diameter of the catheter 702, e.g., the outer diameter 716 of the hub 714 may be, e.g., from about 0.5 mm to about 2 mm, e.g., about 1 mm. Accordingly, in view of the stepped change in outer diameter of the assembly 700 at the distal face 710 of the hub 714, the distal face 710 may act as a depth stop 706, allowing insertion of the microneedle 704 through the RWM until the distal face 710 contacts the RWM, while limiting, resisting, or preventing any further insertion beyond that extent.

The hub 714 may be substantially cylindrical, as shown in, e.g., FIGS. 24-25, or may be angled, as shown in FIG. 26. An angled hub 714 such as shown in FIG. 26 may in certain use cases provide added case of visualization and/or access to the RWM 112.

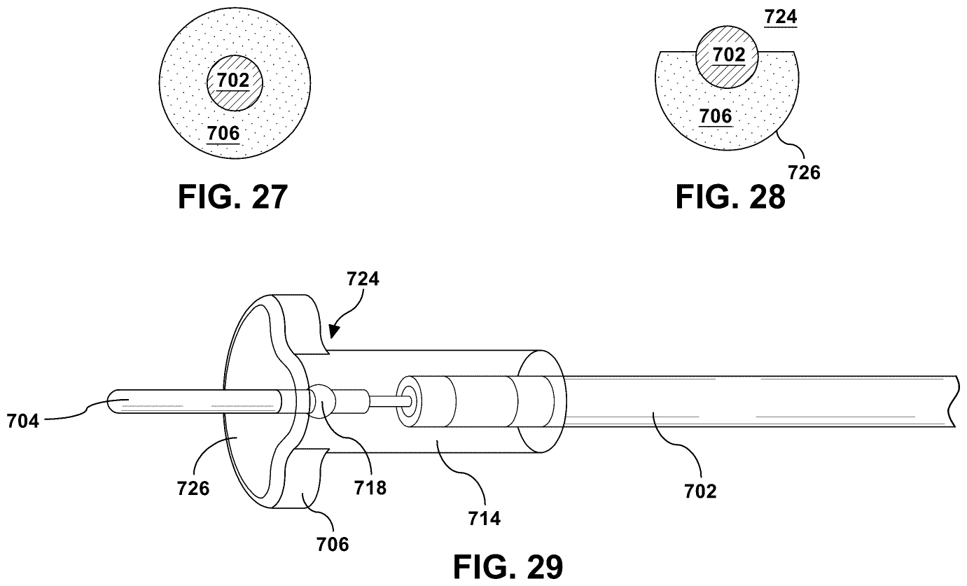

As shown in FIGS. 27-34, in certain embodiments, the hub 714 700 may include a depth stop feature 706, which may be disposed on a distal end of the hub 714. The depth stop 706 may provide a physical feature adapted to abut the RWM in use, when the microneedle 704 has been inserted to a distance corresponding to the desired insertion distance. Upon such contact between the depth stop 706 and the RWM 112, the depth stop 706 may act to limit, resist, or prevent any further insertion of the microneedle 704, and/or to provide tactile feedback, e.g., resistance, to the user indicating that the desired insertion distance has been reached. The depth stop 706 may be made of a rigid or a flexible material, e.g., plastic, silicone, rubber, a thermoplastic material, etc.

The depth stop 706 may have a cross sectional shape adapted to provide a larger surface area, e.g., a larger effective diameter than the distal face 710 of the hub 714 as shown in, e.g., FIGS. 24-26. The hub 714 may include a proximal portion having a first, outer diameter which may be, e.g., about 1 mm, and a distal portion having a second outer diameter that is larger than the first outer diameter, to form the depth stop 706 (e.g., FIGS. 27-34). The second, larger outer diameter may be, e.g., 1-2 mm, 2 mm or greater, and so on. In certain embodiments (e.g., FIGS. 23, 27), the depth stop 706 may have a circular cross-sectional shape, with an outer diameter that is greater than that of the catheter 702. In other embodiments, the depth stop 706 may include various features to provide the functionality of the depth stop 706 while also providing visibility of the needle 704 around the depth stop 706 in use. For example, FIGS. 28-29 illustrate a depth stop having a “half-moon” cross-sectional shape. This embodiment provides a portion or lobe 726 of the depth stop 706 that extends radially outward beyond the circumference of the catheter 702, and a cutaway portion or cutout 724 providing a line of sight between a user positioned proximally relative to the depth stop 706, and the microneedle 704, which is positioned distally relative to the depth stop 706. The cutout 724 may provide, e.g., about 90° to about 270° of visualization about the circumference of the catheter 702, or about 135° to about 205°, or about 180° of visualization in various embodiments. The cross-sectional shape of the depth stop 706 may be curved as shown in FIG. 29, or may include one or more straight edges as shown in FIG. 28.

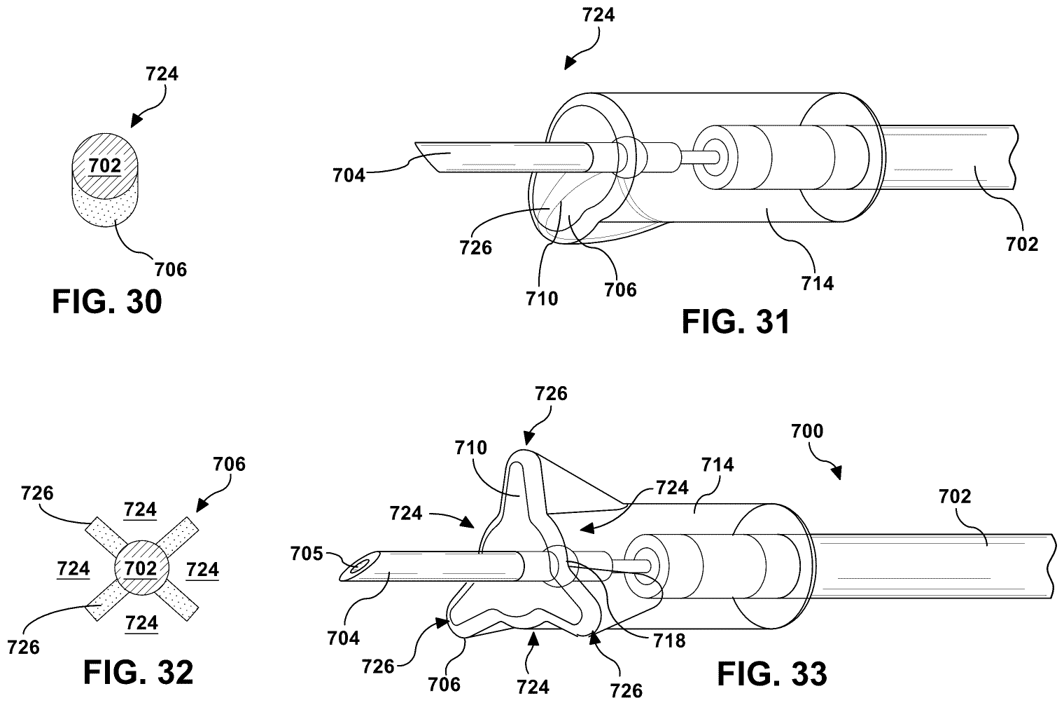

In another example, FIGS. 30-31 illustrate a depth stop 706 having a “rain drop” cross-sectional shape. Like FIGS. 28-29, the depth stop 706 of FIGS. 30-31 provides a portion or lobe 726 of the depth stop 706 that extends radially outward beyond the circumference of the catheter 702, and a cutaway portion 724 providing an unobstructed view and a line of sight between a user positioned proximally relative to the depth stop 706, and the microneedle 704, which is positioned distally relative to the depth stop 706. In this embodiment, the lobe 726 may extend about 180° or less about the circumference of the catheter 702, such that about 180° or more, e.g., about 270° of the circumference of the catheter 702 is open to facilitate visualization of the needle.

FIG. 32 illustrates a depth stop 706 having a “cross” or “x” cross-sectional shape, having four portions, e.g., legs or lobes 726, and four cutaway portions 724 disposed between each of the legs or lobes 726. As shown, the legs or lobes 726 are regularly spaced about the circumference of the catheter 702, each separated by about 90°, but in other cases the legs or lobes 726 may be radially spaced at different or varying intervals. The four cutaway portions 724 provide four potential lines of sight for a user to view the microneedle 704 unobscured by the depth stop 706.

FIG. 33 illustrates a depth stop 706 having a “tripod” cross-sectional shape, having three lobes 726 with three cutaway portions 724 disposed therebetween, with each lobe 726 extending radially outward and approximately equally circumferentially spaced relative to each other lobe 726. The three cutaway portions 724 provide three potential lines of sight for a user to view the microneedle 704 unobscured by the depth stop 706. As a result of the radial extent of each of the legs or lobes 726, the distal surface 710 of the hub 714 of FIG. 33 has a larger effective diameter than the proximal end of the hub 714, thereby providing increased stability in use.

Other numbers of lobes 726 and cutaway portions 724, and other spacing arrangements of the lobes 726 and cutaway portions 724 may also be used. For example, various embodiments may include one lobe, two lobes, three lobes, four lobes, five lobes, or more than five lobes, separated by one, two, three, four, five, or more than five cutaway portions, and so on.

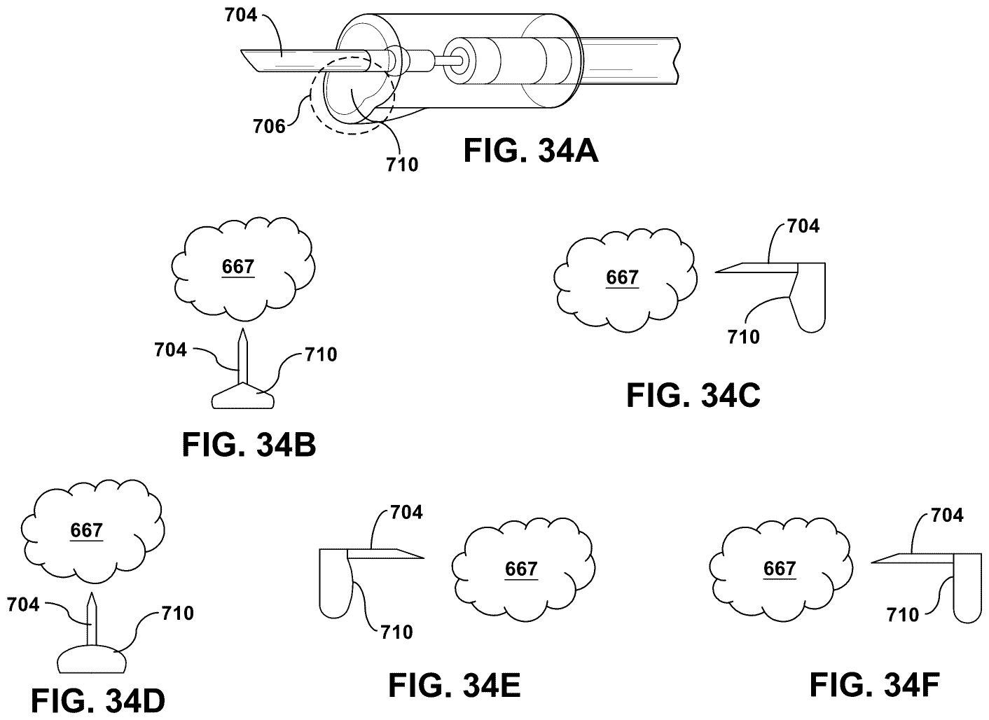

In certain embodiments, the distal face 710 of the depth stop 706 may be flat or substantially flat, as shown in, e.g., FIG. 33. However, with reference to FIG. 34, in other embodiments, the distal face 710 may be, e.g., angled or convex to allow for various vector insertions of the catheter assembly 700. FIGS. 34B and 34C illustrate bottom and side views, respectively, of an embodiment in which the distal face 710 is angled, having a raised point in or near the center of the distal surface 710. FIGS. 34D and 34E illustrate bottom and side views, respectively, of a further embodiment in which the distal face 710 is convexly shaped. These angled and convex depth stops may be useful to protect delicate features of the car in use cases in which an approach along a particular vector is used. FIG. 34F illustrates a corresponding side view of an embodiment in which the distal face 710 is flat.

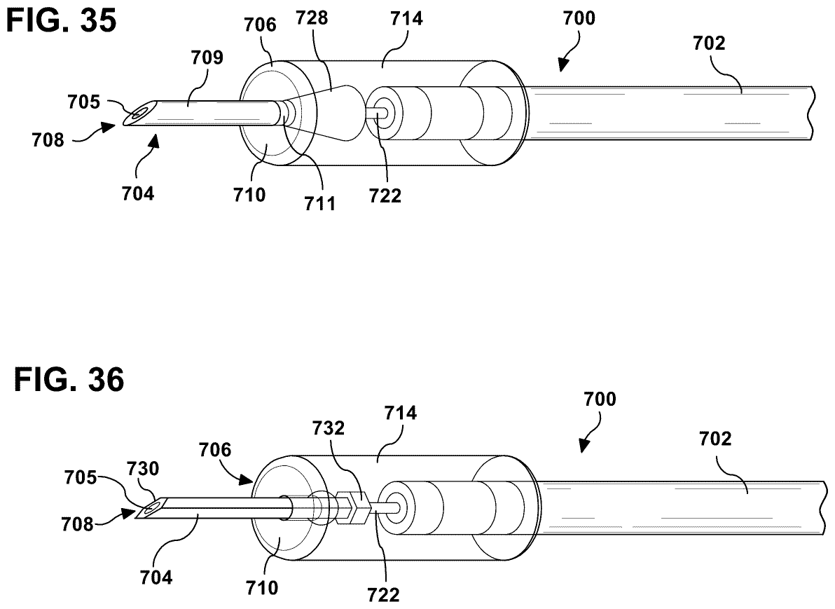

Referring to FIG. 35, in certain embodiments the microneedle 704 may include a retention feature in the form of a proximal end portion 728 having a larger diameter than that of the microneedle 704 shaft. The proximal end portion 728 may be, e.g., flared, such that the outer diameter of the microneedle 704 increases at the proximal end thereof. In such embodiments, the microneedle 704 shaft 709 and the distal tip 708 may maintain a consistent or substantially consistent maximum outer diameter. For example, where the microneedle 704 is a 32-gauge needle, the outer diameter of the shaft 709 may be about 0.235 mm. Progressing proximally from a point 711 along the axial extent of the microneedle 704, at which the microneedle 704 transitions from the shaft 709 to the proximal end portion 728, the outer diameter of the microneedle 704 progressively increases to produce a flared geometry. The transition point 711 may be disposed within the hub 714 or near the distal end of the hub 714. The hub 714 may be insert molded with the microneedle 704, such that the flared proximal end portion 728 may be partially or entirely disposed within the hub 714. In this manner, the flared geometry of the proximal end portion 728 contributes to the mechanical interlock between the microneedle 704 and the hub 714, limiting, e.g., preventing translation of the microneedle 704 relative to the hub 714.

Referring to FIG. 36, the microneedle 704 may in certain embodiments be a silicon needle. Such microneedles may be formed by “growing the needle in silicon,” e.g., using nanotechnology processes such as, e.g., etching, chemical vapor deposition (CVP), etc. to fabricate the needles. The silicon microneedle 704 shown in FIG. 36 embodies a hexagonal cross-sectional geometry. Use of a silicon microneedle 704 in the otic catheter assembly 700 provides a sharp tip 708 with a relatively short bevel 730 compared to conventional metal needles of similar size and sharpness. The silicon microneedle 704 may include a mechanical interlock feature 732 having a hexagonal cross-sectional shape, disposed at or near the proximal end of the microneedle 704. The mechanical interlock feature 732 need only have an outer diameter that exceeds that of the microneedle shaft. The silicon microneedle 704 may be insert-molded with the hub 714, and the mechanical interlock feature 732 may be disposed within the hub 714 such that the mechanical interlock feature 732 contributes to the fixation of the microneedle 704 within the hub 714, thereby limiting, e.g., preventing translation of the microneedle 704 relative to the hub 714.

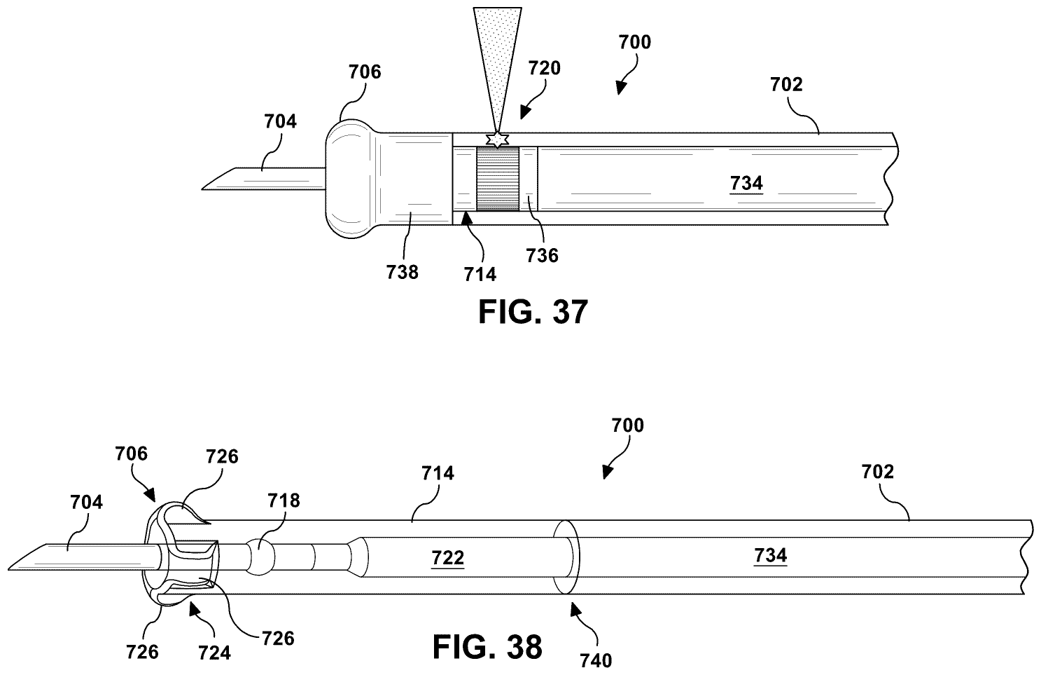

According to an embodiment depicted in FIG. 37, the otic catheter assembly 700 includes a hub 714 that is at least partially inserted into the lumen 734 at the distal end of the catheter 702. The hub 714 may include a proximal portion 736 having a diameter selected to be received with a close fit within the lumen 734 of the catheter 702. The distal portion 738 may have an outer diameter that is larger than that of the proximal portion 736. The distal portion 738 may further include a depth stop 706 having an outer diameter that is greater than the outer diameter of the distal portion 738, and may also be greater than the outer diameter of the catheter 702. The depth stop 706 depicted in FIG. 37 is round in cross section, although any geometry or configuration, such as those described elsewhere herein relative to, e.g., FIGS. 27-34, may be used. The hub 714 may be, e.g., insert-molded about a microneedle 704, and may be made of, e.g., an opaque plastic material, while the catheter 702 may be made of, e.g., a translucent or transparent plastic material. The hub 714 may further be affixed in an axial position relative to the catheter 702 by a weld 720, e.g., a radial laser weld.

FIG. 38 illustrates an embodiment including a hub 714 to provide integration of the microneedle 704 with the catheter 702. Various components, e.g., the hub 714 and the catheter 702, may be affixed to one another using welding techniques such a butt weld. As shown in FIG. 38, the catheter assembly 700 includes a microneedle 704 and a hub 714, which may be insert-molded about the microneedle 704 as described previously, and a catheter 702 made of a material that is the same as that of the hub 714. The hub 714 may include a lumen 722 that extends therethrough. The lumen 722 may transition in inner diameter from a first inner diameter at a distal end that corresponds, or approximately corresponds to the outer diameter of the microneedle 704, to a second, larger inner diameter at a proximal end of the hub 714. In certain embodiments, the second, larger inner diameter at the proximal end of the hub 714 may correspond, or approximately correspond, to the diameter of the lumen 734 of the catheter 702. The proximal end of the hub 714 may be adjacent to, or abut the distal end of the catheter 702. The proximal end of the hub 714 and the distal end of the catheter 702 may be joined together by a butt welding process such as, e.g., radio frequency (RF) welding.

As further shown in FIG. 38, the hub 714 may include a lobed depth stop 706, including features similar to those described relative to FIG. 33. In particular, the depth stop 706 may have a cross sectional shape adapted to provide a larger effective diameter, while also providing visualization of the microneedle 704 from multiple angles and along multiple vectors by the user. The depth stop 706 may include one or more cutaway portions 724 disposed about or between one or more lobes 726. In use, the user may visualize the microneedle 704 through any of the cutaway portions 724, providing visualization advantages associated with a smaller diameter hub 714 and depth stop 706, while the lobes 726 provide the stability associated with a larger diameter hub 714 and depth stop 706. The embodiment depicted in FIG. 38 includes three lobes 726 with three cutaway portions 724 disposed therebetween, with each lobe 726 extending radially outward and approximately equally circumferentially spaced relative to each other lobe 726. This configuration results in a depth stop 706 having a tripod-shaped cross-section. However, other numbers of lobes 726 and cutaway portions 724 and other spacings of the lobes 726 and cutaway portions 724 may also be used.

FIGS. 39-40 illustrate embodiments of an otic catheter assembly 700 including a microneedle 704 and a catheter 702 disposed directly thereon, i.e., without a hub 714 disposed therebetween. The microneedle 704 may be paired with a catheter 702 having an inner diameter that corresponds to, or nearly corresponds to the outer diameter of the microneedle. For example, a 32-gauge microneedle 704 may be paired with a 1.5 French catheter. The catheter 702 may be made of, or include a jacket (such as jacket 202, FIGS. 4, 8B) that is made of a firm or a flexible material, and may be uniform in firmness or segmented along the axial extent of the catheter. A heat swaging or thermoforming process may be used to soften the catheter material and squeeze the distal end 742 of the catheter 702 over the proximal end of the microneedle 704 in a sleeve-like fashion. The catheter 702 may be rolled or reformed over the microneedle 704, providing a precise and tight fit over the proximal end of the microneedle 704 and features thereof. As illustrated, the microneedle 704 may include a retention feature such as, e.g., a bead 718 disposed on the microneedle at or near a proximal end thereof. The catheter 702 may be formed over the bead 718 or similar feature, such that the catheter 702 fits tightly thereover, and provides a tight fit or seal. The distal edge 744 of the catheter 702, disposed over the microneedle 704 distally of the bead 718 or other retention feature, may act as a depth stop, limiting the extent of insertion of the microneedle 704 through the RWM 112.

FIG. 41 illustrates an otic catheter assembly 700 including a catheter 702, a microneedle 704, and a hub 714. The hub 714 may be a substantially annular component, having a “C” shaped cross section as shown in FIG. 41. The hub 714 may also be described as a collar or as being “cup shaped,” or as a partially hollowed cylinder. A bore 746 may extend longitudinally through the hub 714, the bore being dimensioned at the proximal end thereof 748 to accommodate the outer diameter of the catheter 702 with a close fit. The longitudinally extending bore may be dimensioned at the distal end of the bore 750 to accommodate the outer diameter of the microneedle 704 with a close fit, but to not accommodate the outer diameter of the catheter 702.

The catheter 702 may be selected such that the inner diameter of the lumen 734 of the catheter 702 may receive and accommodate the outer diameter of the microneedle 704. The proximal end of the microneedle 704 may be disposed within a portion of the lumen 734 that is disposed within the hub 714. Due to the closeness of the fits between each contacting pair of components, the distal end of the bore 750 does not accommodate the outer diameter of the catheter 702.

The hub 714 of FIG. 41 may be disposed over the microneedle 704 such that the distal end of the bore 750 of the hub 714 is disposed about the microneedle 704. The hub 714 may be made of a material including a metal, and may be affixed to the outer diameter of the microneedle 704 by welding, brazing, or other metal joining technique as understood by one of skill in the art. The distal end of the catheter 702 may then be inserted into the proximal end of the bore 748 in the hub 714, until the distal edge 744 of the catheter 702 contacts the proximal face 752 of the distal end of the hub 714.

In one embodiment, a heat joint may be formed, wherein the catheter 702 is pressed distally into the socket formed by the hub 714 with heat as the material, e.g., plastic of the catheter 702 flows. The melted material, e.g., plastic of the distal end of the catheter 702 may flow within the hub 714 as shown by the arrows 754 and about the proximal end of the microneedle 704, within an opening 756 within the hub 714 between the proximal and distal bore openings 748, 750. As the melted material, e.g., plastic flows within the opening 756, a retention feature is created within the hub 714, which cannot be pulled proximally out of the hub 714 due to the smaller diameter of the bore 748 at the proximal end of the hub. Alternatively, instead of a heat joint, the hub 714, which may be made of metal, may be crimped or formed radially inward, e.g., using a tool, thereby squeezing the distal end of the catheter 702, and resulting in the secure retention of the proximal end of the microneedle 704 within the distal end of the catheter 702.

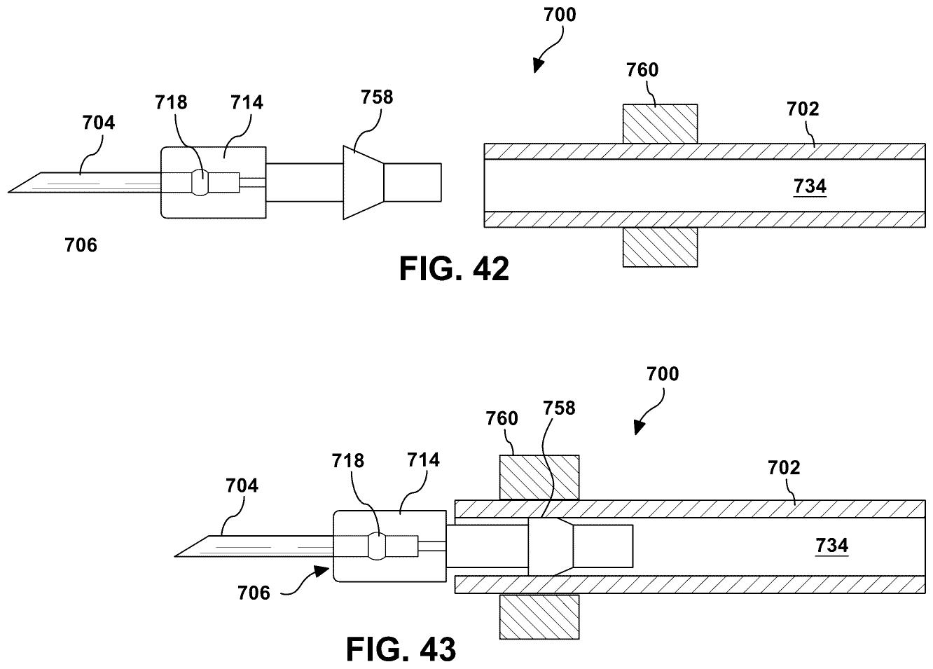

Turning to FIGS. 42-43, an otic catheter assembly 700 is illustrated, including a catheter 702, a hub 714, and a microneedle 704. As shown, the hub 714 may be insert-molded on the needle 704, which may include a bead 718 or other retention feature as described herein. A barb 758 may be provided as a feature of the hub 714 as shown, or may alternatively be formed on the microneedle 704. The catheter 702 may then be pressed onto the proximal end of the hub 714, including the barb 758, as shown in FIG. 43, such that the hub 714 or a portion thereof including the barb 758 is disposed within the lumen 734 of the catheter 702. In addition to the resistance to axial movement provided by the barb 758, the position of the hub 714 may be further secured by an external crimp, collar, or clamp such as clamp 760 disposed about an exterior surface of the catheter 702. The clamp 760 or crimp may be disposed over the barb 758, providing additional retention force. In further embodiments, the fit between the catheter 720 and the hub 714 may be additionally or alternatively secured by heat shrinking, or through use of a press fit ring.

With reference to FIGS. 44-49, various needle features are shown, including, e.g., anti-coring features and bent tip needles. Anti-coring features may allow for the creation of a smaller, less invasive incision in tissue such as the RWM in use, and faster healing times for patients after, e.g., intracochlear infusion procedures. For example, FIG. 44 illustrates a Quincke needle tip, including a cutting bevel 730 having a length 762 and a sharp tip 708 providing access to a lumen 705. FIGS. 45-47 illustrate needles 704 featuring a single lateral orifice 764 and an elongated pencil point tip 708 such as, e.g., Sprotte and Whitacre needles. The orifice 764 may be formed, e.g., via laser, wire electrical discharge machining (EDM), grinding, electrochemical grinding, or etching, and provides access to the lumen of the needle. FIG. 48 illustrates features of a Tuohy needle, which includes a slight curve 766 at the distal tip 708, while FIG. 49 illustrates a needle 704 having a bent tip 768.

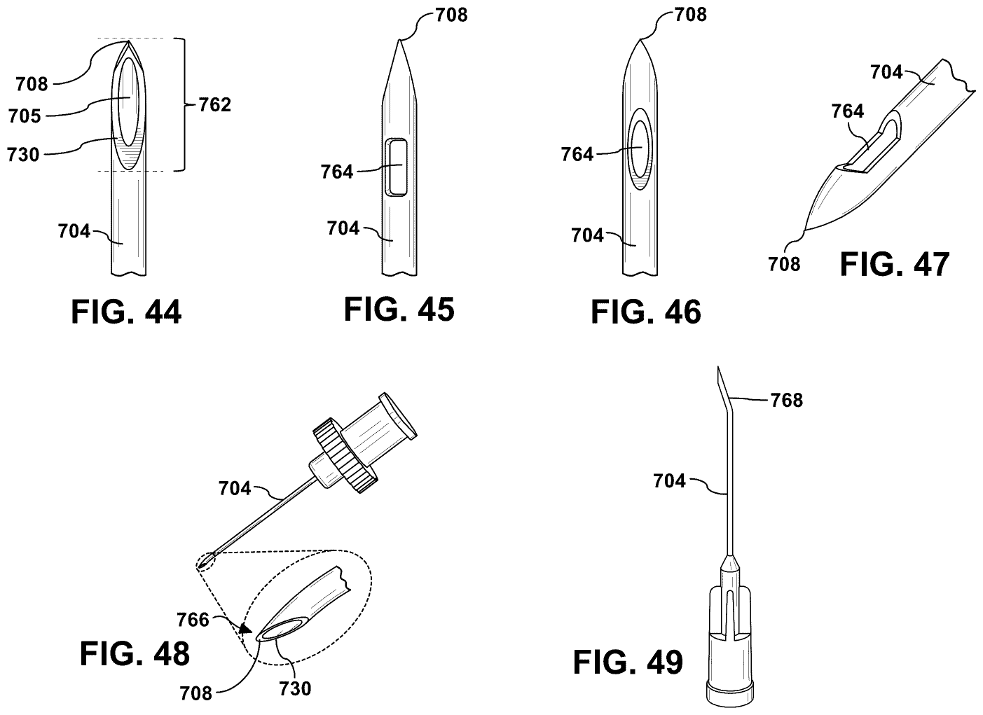

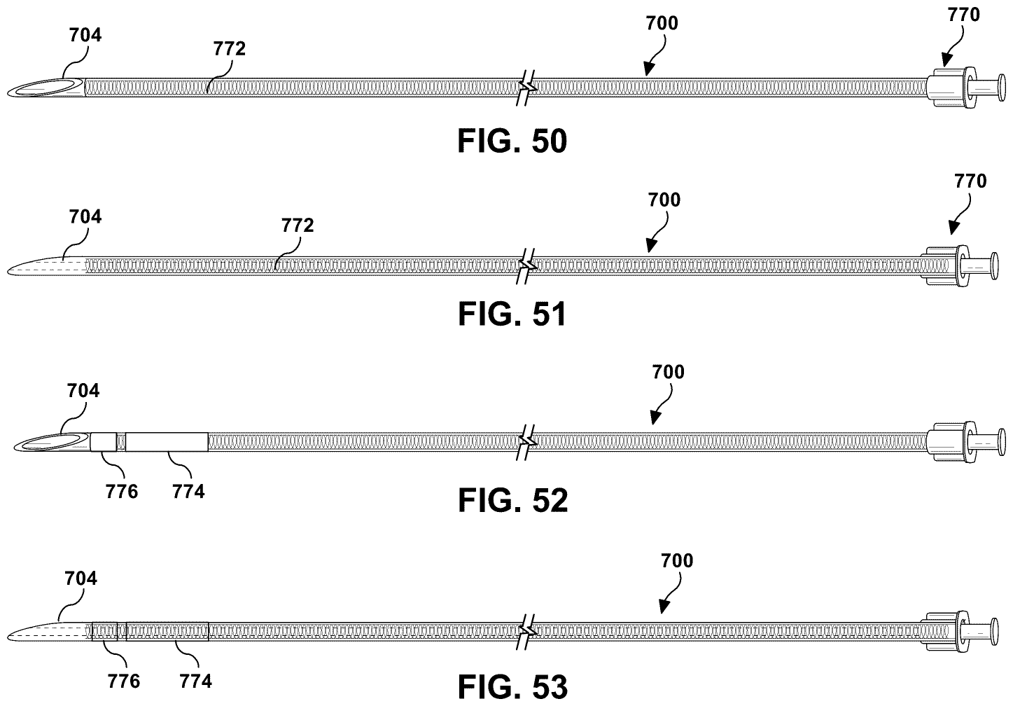

Turning next to FIGS. 50-53, a number of features of catheter 702 may be provided for use in the otic catheter assembly 700. With reference to FIGS. 50-51, a luer lock connection 770 may be provided for fluidly and securely connecting the proximal end of the catheter 702 to, e.g., a syringe containing the drug product to be infused into the cochlea. In certain embodiments, the catheter 702 may also include a needle 704 welded thereto. This may provide a simplified and streamlined device for use. Additionally, the catheter may be spring-guided, e.g., by spring 772. In particular, the spring 772 may bias the catheter in the distal direction in use.

In certain embodiments, with reference to FIGS. 52-53, the otic catheter assembly 700 may include visual indicators such as, e.g., ink markings, to guide the user during use. For example, markings 774 may be provided to guide the user on where to hold the catheter 702 during use, to promote structural integrity and to limit, e.g. prevent buckling or drooping of the catheter 702 as it is navigated to and into the insertion site. Additionally or alternatively, markings 776 may be provided to guide the user regarding the correct depth to which the assembly should be inserted through the RWM. For example, the correct depth may be, e.g., 5 mm or less, 4 mm or less, 3 mm or less, 2 mm or less, or 1 mm or less, which may correspond to the location of the marking 776. Markings 774, 776 may cover the periphery of the catheter 702, i.e., may extend 360° around the outer surface thereof, or may only extend partway around the circumference of the catheter 702. Markings 774, 776 may further be deployed in combination with any embodiments described herein, including but not limited to those including a depth stop 706.

With reference to FIGS. 54-57, certain embodiments of the disclosure provide an otic catheter assembly including one or more features adapted to provide the device with shape memory. For example, FIGS. 54-55 illustrate portions of an otic catheter assembly 800 having a catheter 802 and a depth stop 804 similar to, e.g., depth stops 232 or 706 as described herein above. A first shape memory feature 806 or set thereof (e.g., shape memory feature(s) 806) may be disposed on, and extend proximally along the catheter 802 from the depth stop 804. In certain embodiments including a first set of shape memory features 806, two or more shape memory features 806 may be provided, e.g., two, three, or more shape memory features 806, each of which may be spaced apart from each other shape memory feature 806 about the circumference of the catheter 802. For example, where two shape memory features 806 are provided in the first set of shape memory features 806, as shown in FIG. 54, the shape memory features 806 may be spaced about 180° from one another about the circumference of the catheter 802.

The first shape memory feature 806 or set thereof may extend about 5 mm to about 15 mm, e.g., about 10 mm in a proximal direction from the depth stop 804. The first shape memory feature(s) 806 may be, e.g., a flat or round wire, and may be made of, e.g., nitinol or a metal. The shape memory feature(s) 806 may be adapted to selectively provide rigidity to the portion of the catheter 802 immediately proximal of the depth stop 804, corresponding to the length of the first shape memory feature(s) 806. The rigidity of this section may be adapted to limit or prevent the formation of undesirable bends, twists, movement, or kinks in the catheter 802 in use. The shape memory feature(s) 806 may be further adapted to be selectively bent, angled, or flexed into a desired configuration by a user, and to maintain the desired configuration during insertion into the RWM and delivery of the infusion. Such features may enhance navigability of the distal tip of the catheter assembly 800 into the RWM in use.

In certain embodiments, as shown in FIG. 55, the shape memory feature(s) 806 may be disposed on an outer surface, or embedded within the walls of an inner sheath 808 similar to sheath or liner 204 of FIG. 4. As shown in FIG. 54, a jacket or overcoating 810 (similar to jacket or overcoating 202 of FIG. 4) may be disposed thereover. As shown in FIGS. 54 and 55, a rigid layer 812 may be disposed about the inner sheath 808 and within the jacket or overcoating 810. Like the rigid layer 208 (see, e.g., FIGS. 4, 7A-7B, 8A-8B, and others) discussed herein above, the rigid layer 812 may include, e.g., a coil, a spring, a mesh, or a braid made of metal such as, e.g., stainless steel or another non-metal material, and may be adapted to provide rigidity and structural support to the catheter assembly 800.

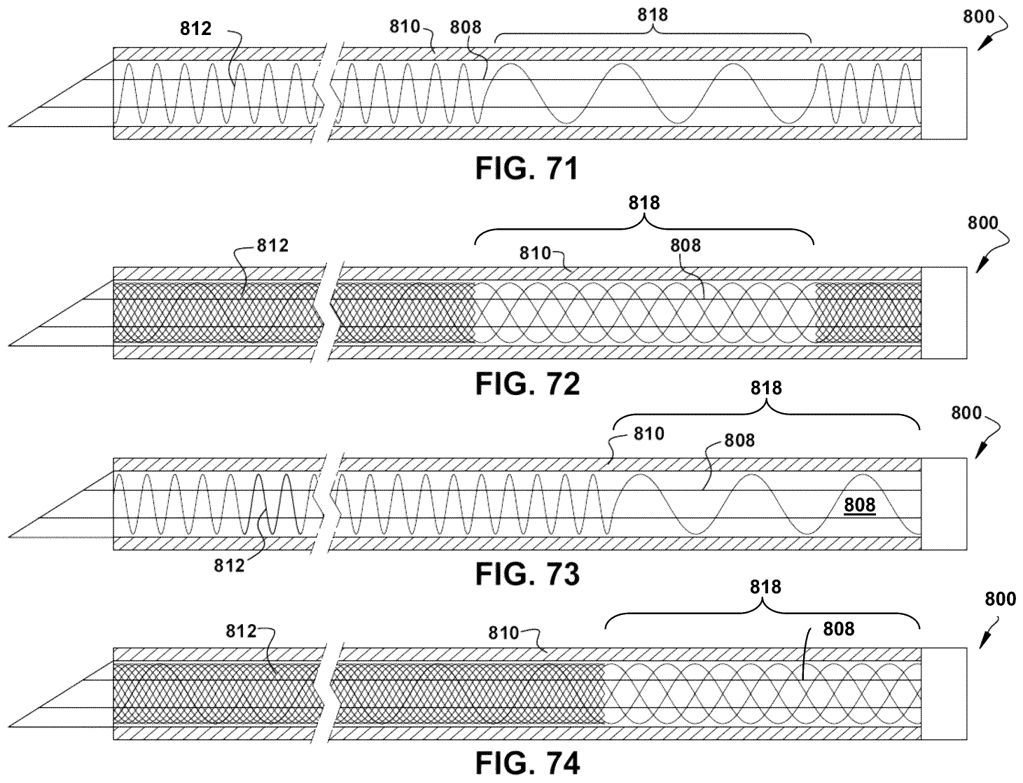

As shown in FIGS. 71-74, in certain embodiments, the rigid layer 812 may include a priming window through which the drug product may be visualized through the rigid layer 812. For example, where the rigid layer 812 is a spring (FIGS. 71, 73), the pitch of the spring may be varied, such that the pitch is greater, i.e. the distance between coil centers is further apart in certain regions than others. In regions where the pitch is greater, i.e., the coil centers are further apart, a priming window 818 is provided in which a user may visualize the fluid path of the drug product between the coils of the spring, which are present at a lower density in the priming window than in other regions along the axial extent of the catheter assembly 800. Where the rigid layer 812 is a braid (FIGS. 72, 74), the pitch or spread of the braid may be similarly varied, such that the braid is more open in certain regions than others. In regions where the pitch or spread is greater, i.e., the braid is looser, a priming window 818 is provided in which a user may visualize the fluid path of the drug product through the braid.

The inner sheath 808 and overcoating 810 (where present) may be made of one or more transparent or translucent materials, which permit sufficient visibility for a user to assess the presence or absence of bubbles in the drug product, as it flows through the catheter assembly 800 past the priming window 818. For example, the inner sheath 808 and overcoating 810 may be made of thermoplastic materials which may be transparent or substantially transparent. Alternatively, the inner sheath 808 may be made of a transparent or substantially transparent material, and the overcoating 810 may be made of an opaque or substantially opaque material (e.g., nylon-based), and may be partially or completely removed or absent from the axial extent of the catheter assembly 800 in which the priming window 818 is located. The presence of one or more bubbles in the drug product, viewed through the priming window 818, would indicate a need for further priming prior to administration. On the other hand, visual confirmation of the absence of bubbles in the drug product visible in the priming window may indicate that the device is primed and ready for administration.

As shown in FIGS. 71-72, in certain embodiments, the priming window 818 may be located along the axial extent of the catheter assembly 800, rather than at the proximal or distal end thereof. The relatively smaller pitch of the rigid layer 812 at the proximal and distal ends provides additional stiffness to the proximal and distal ends, and to the syringe connection and insertion end, respectively. In other embodiments as shown in FIGS. 73-74, the priming window 818 may be positioned near or adjacent the proximal end of the catheter assembly 800. This positioning may facilitate prompt observation of any bubbles entering the catheter assembly 800, indicating a need for additional priming, before the drug product has traveled the length of the catheter assembly 800.

Referring back to FIG. 56, in certain embodiments, the shape memory feature(s) 806 may be affixed to the rigid layer 812. For example, the shape memory feature(s) 806 may be tack welded to the rigid layer 812 at, e.g., fixation points 814. As shown, the rigid layer 812 may be, e.g., a coil spring, and at fixation points 814, individual coils of the rigid layer 812 may be affixed to the shape memory feature(s) 806. A jacket 810 may be disposed about or over the rigid layer 812, the shape memory feature(s) 806, and the liner or sheath 808, as shown in FIG. 54.

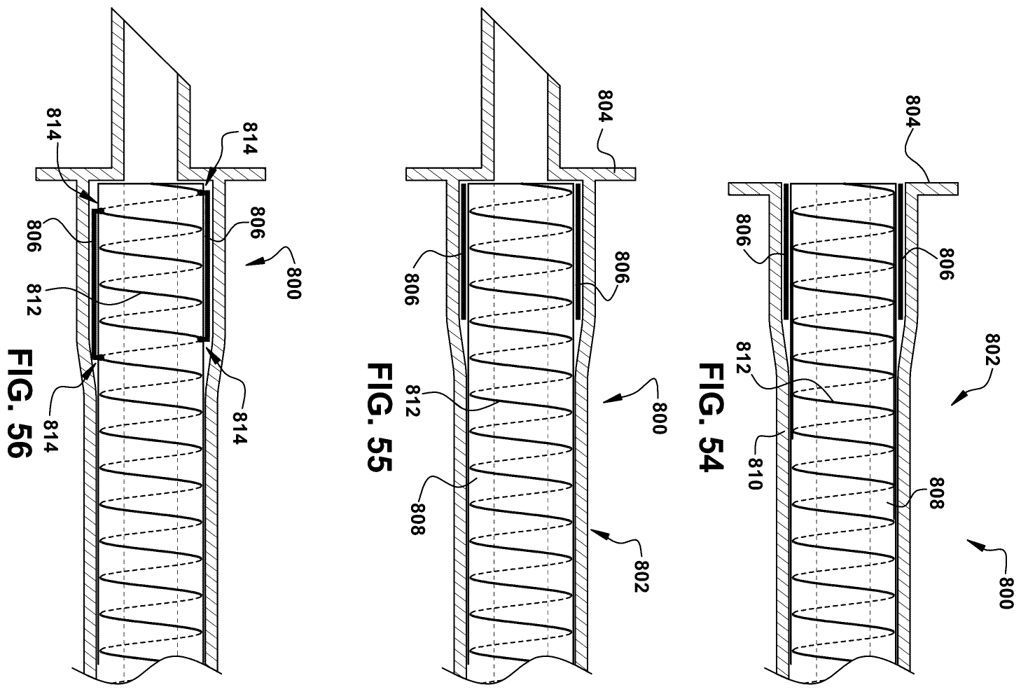

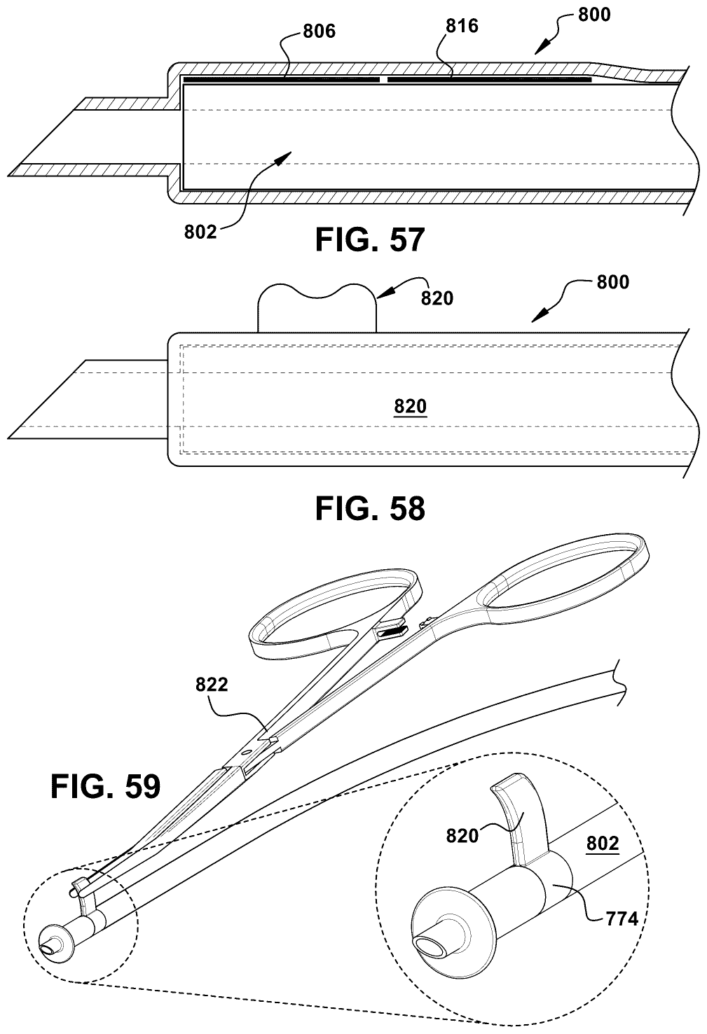

In certain embodiments, such as shown in FIG. 57, a second shape memory element 816 or set thereof (e.g., shape memory element(s) 816) may be disposed proximally of the first shape memory element(s) 806. In certain embodiments, the second set of shape memory element(s) 816 may be of similar length to the first set of shape memory element(s) 806, and may be adjacent to (e.g., positioned proximally relative to) the first shape memory element(s) 806. In other embodiments, the second set of shape memory element(s) 816 may be axially spaced from the first set of shape memory element(s) 806. In certain embodiments including a second set of shape memory features 816, two or more shape memory features 816 may be provided, e.g., two, three, or more shape memory features 816, each of which may be spaced apart from each other shape memory feature 816 about the circumference of the catheter 802. Additionally, the number of shape memory features 816 in the second set of shape memory features 816 may be selected independently from the number of shape memory features 806 in the first set thereof. Accordingly, certain embodiments may include, e.g., a first set of two shape memory elements 806, and a second set of one or two shape memory elements 816. The shape memory features 816 of the second set may be spaced about the circumference of the catheter 802 in a manner similar to that described with respect to the first set 806 of shape memory features, and may or may not be aligned therewith.

The second shape memory feature(s) 816 or set thereof may have an axial length of about 5 mm to about 15 mm, e.g., about 10 mm, similar to the shape memory feature(s) 806 of the first set. The second set of shape memory feature(s) 816 may be, e.g., a flat or round wire, and may be made of, e.g., nitinol or a metal. The shape and material of the second set of shape memory feature(s) 816 may be selected independently from that of the first shape memory feature(s) 806. The first and second sets of shape memory feature(s) 806, 816 may be adapted to selectively provide rigidity to the portion of the catheter 802 immediately proximal of the depth stop 804 corresponding to the length of the first shape memory feature(s) 806 and 816. In certain embodiments, the shape, thickness, and/or material of the second set of shape memory feature(s) 816 may be different from that of the first set of shape memory feature(s) 806, thereby providing segmented and/or variable rigidity to the device.

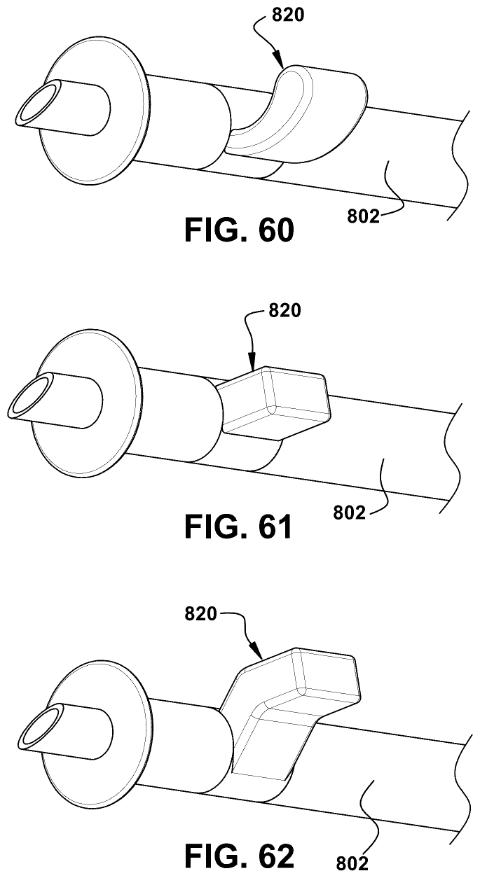

Turning to FIGS. 58-62, certain embodiments of the disclosure provide an otic catheter assembly including one or more wing 820 features adapted for grasping using a surgical instrument, e.g., forceps 822, to position the catheter assembly during use. As shown in FIG. 58, the wing or flag 820 may extend radially outward from the outer surface of the catheter assembly 800, e.g. from the outer surface of catheter 802. In certain embodiments, as shown in FIG. 59, the wing 820 may be axially aligned with a visual marking 774. As described herein above, the marking 774 may be provided to guide the user on where to hold the catheter during use in order to promote structural integrity and to limit, e.g. prevent buckling or drooping of the catheter 702 as it is navigated to and into the insertion site. The wing 820 may be, e.g., curved (FIG. 60) in shape, may extend at a right angle (FIG. 61), or may include an angled portion (FIG. 62) relative to the longitudinal axis of the catheter 802, or adopt any other conformation as will be understood by one of skill in the art.

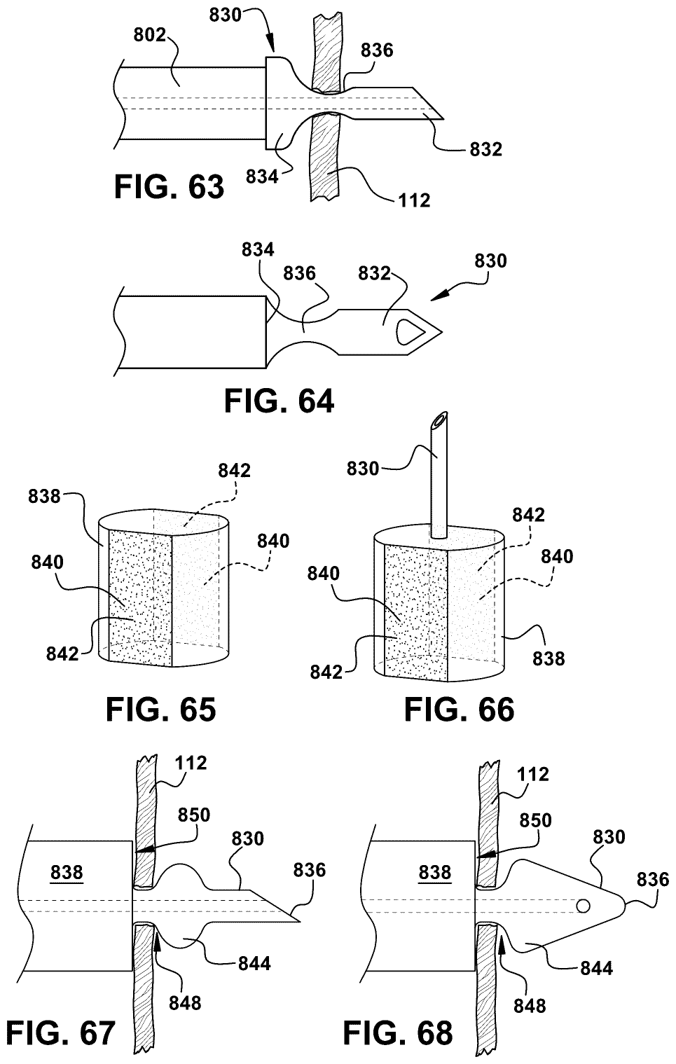

Turning to FIGS. 63-69, certain embodiments of the disclosure provide an otic catheter assembly including a needle 830 having a cross sectional shape and dimensions that vary along the axial extent of the needle 830. These variations are adapted to support and maintain a position of the needle within the RWM 112 during a procedure such as, e.g., an infusion. For example, as shown in FIG. 63, the needle 830 may include a curved or hourglass shape, illustrated in cross section, in which the distal portion 832 and proximal portion 834 of the needle 830 each have a greater diameter than that of a central portion 836 disposed therebetween. In use, the needle 830 may be inserted through the RWM 112 to a pre-determined distance, and at this predetermined insertion distance, the RWM 112 is disposed about the central portion 836 having the relatively smaller diameter. The increase in diameter as the needle transitions from the central portion 836 to the proximal portion 834 tends to resist further insertion of the needle 830 into the RWM 112, while the increase in diameter as the needle transitions from the central portion 836 to the distal portion 832 tends to resist withdrawal of the needle 830 from the RWM 112, e.g., via passive movements. While the profile of the needle 830 does not prevent or preclude insertion of the needle 830 through the RWM 112, it tends to protect cochlear anatomy by resisting unintended movements mid-infusion procedure. FIG. 64 illustrates a needle 830 having a similar geometry, including a central portion 836 disposed between a distal portion 832 and a proximal portion 834, each of which has an outer diameter greater than that of the central portion 836. The needle 830 of FIG. 64 includes curves and a smooth section to minimize damage to the RWM and local anatomy upon withdrawal of the needle 830 from the RWM.

FIGS. 65-67 illustrate embodiments in which the needle 830 is formed integrally with a hub 838. In one embodiment, shown in FIGS. 65-66, the hub 838 may include features adapted to facilitate grasping, e.g., using forceps. For example, the hub 838 may include flat surfaces 840 extending longitudinally along the hub 838 on either side thereof, which may further be textured for additional friction. Each of these features may be adapted to increase control over the device during a procedure.

In further embodiments, shown in FIGS. 67-68, the needle 830 may include a bulge 844, or area of increased diameter at a point along the axial extent thereof. FIG. 67 illustrates a Quincke needle, and FIG. 68 illustrates a pencil point needle, each with a bulge 844. The bulge 844 may be disposed proximally relative to the distal tip 846 of the needle 830, and may be positioned axially such that in use, the RWM 112 is disposed immediately proximally of the bulge 844. In this manner, the bulge 844 acts as a shoulder, resisting movement of the needle 830 in the proximal direction after its insertion into the RWM 112 to a desired distance. The desired distance of insertion corresponds to a distance from the distal tip 836 of the needle 830 to the proximal face 848 of the bulge 844. Additionally, the distal face 850 of the hub 838 may be adapted to resist movement in the distal direction after its insertion into the RWM 112 to the desired distance. According to certain embodiments, the space between the distal face 850 and the proximal face 848 may be dimensioned to accommodate and retain the RWM 112, e.g., during the duration of an infusion procedure. In each of the foregoing embodiments, the outer diameter of the bulge 844, although larger than that of the needle 830 shaft, is sufficiently small as to allow insertion of the needle 830 including the bulge 844 through the RWM 112.

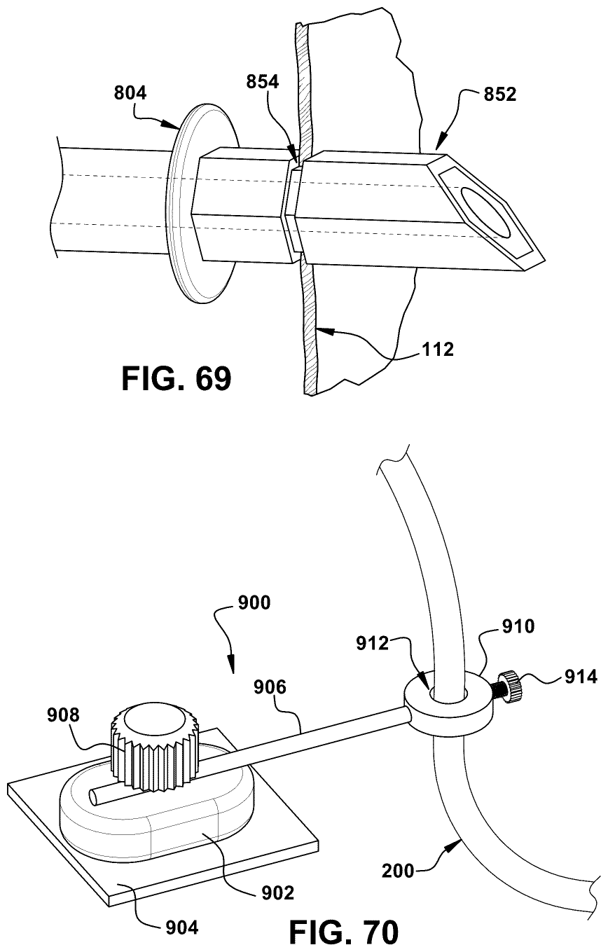

In a further embodiment, FIG. 69 illustrates a further needle 852 which may be used in combination with other aspects of the disclosure. The needle 852 may include a gap or cutout 854 located and dimensioned to accommodate the RWM 112 in use. The gap or cutout 854 may be a portion of the needle 852 having a reduced outer diameter. The reduction in outer diameter may be partial or complete, e.g., it may extend 360° around the circumference of the needle, or it may be partial and may extend fewer degrees around the needle circumference. The cutout 854 may have an axial extent along the needle 852 dimensioned to accommodate the thickness of the RWM 112. In use, the needle 852 may be inserted through the RWM 112 until the RWM 112 is received within the cutout 854. The RWM 112 main remain within the cutout 854 during administration of the infusion, thereby contributing to the stability of position of the needle 852 during the procedure. The reduction in diameter at cutout 854 may be sufficient to resist translation of the needle 852 relative to the RWM 112, but not so great as to result in damage to the RWM 112 or surrounding anatomy upon removal, or to prevent such removal at the conclusion of the procedure. In certain embodiments, the needle 852 may be, e.g., a silicon needle, having a hexagonal cross section shape.

The microneedles described herein may be fabricated in a number of different ways. As described herein, the needles themselves may be metal needles of, e.g., 28 gauge or smaller, or may be silicon needles (FIGS. 36, 69) or micro-molded needles made of thermoplastic material. The needles, regardless of type, may be over-molded to form a hub disposed about the needle. Alternatively, the hub may be omitted in certain embodiments, allowing the needle and the catheter to interface directly. In still further embodiments, as depicted in, e.g., FIGS. 63-68, the needles may be formed using, e.g., micro molding techniques, using a very hard plastic material, e.g., thermoplastic materials. Such fabrication techniques may facilitate the manufacturing of needles such as needle 830 having particular features as described herein, such as the central portion 836 (FIGS. 63-64), hub 838 (FIGS. 65-68), flat surfaces 840 (FIGS. 65-66), bulges 844 (FIGS. 67-68).

Turning to FIG. 70, a catheter support device 900 is provided for use in connection with any catheter assembly described herein such as, e.g., catheter assembly 200, 700, or 800. In use, once the catheter is placed through the RWM and the infusion begins, it is desirable to limit any movement of the catheter, particular the distal-most portion that is inserted through the RWM and is located within the cochlea. Here, even small movements of the catheter may either physically touch anatomical structures within the cochlea, or may disrupt fluid flow patterns within the cochlea, either of which may result in damage to delicate anatomical structures within the cochlea. Accordingly, the support device 900 may be used to position and support the catheter 200, 700 during an infusion procedure, and to maintain a steady and stable position of the catheter 200, 700, 800 during the procedure. The support device 900 may include a body 902 adapted for placement on the skin of a patient at a position near the car, and adhesive, e.g., an adhesive patch 904 disposed on a patient-facing surface of the body 902. In other embodiments, the body 902 of the support device 900 may be adapted for placement on the bone, e.g., skull, of a patient at a position near the car. The body 902 of the support device 900 may be removably affixed to the bone with a fixator such as, e.g., a screw.

A boom 906 may be adjustably affixed to the body 902, and may be adapted for adjustment using one or more boom adjustment actuators 908. Although only one boom adjustment actuator 908 is shown in FIG. 70, certain embodiments may include two or more boom adjustment actuators. Each adjustment actuator 908 may adjust a position of the boom 906 along one or more axes, e.g., an X axis, a Y axis, and/or a Z axis. In various embodiments, the boom adjustment actuator 908 may be, e.g., a knob as shown, or another actuator such as, e.g., a button, lever, switch, toggle, dial, or other control. In use, the position of the boom 906 may be adjusted relative to the body 902, and therefore also relative to the patient anatomy when the adhesive patch 904 is affixed to the patient, to locate the boom head 910 at a desired position relative to the RWM. The boom head 910 may include an aperture 912 therein, through which the catheter, e.g., catheter 200 700, or 800 may be disposed. The boom head 910 may further include a catheter adjustment actuator 914 adapted to actuate translation of the catheter, e.g., catheter 200 700, or 800 in a distal or proximal direction. In various embodiments, the catheter adjustment actuator 914 may be, e.g., a knob as shown, or any other actuator such as, e.g., a button, lever, switch, toggle, dial, or other control. The adjustment actuator 914 may also be adapted to fix or secure an axial position of the catheter, e.g., catheter 200, 700, or 800 when a desired position is attained. Accordingly, the support device 900 may be adapted to achieve a desired position of the catheter, e.g., catheter 200, 700, or 800 for commencement of an infusion procedure, and to maintain such a position throughout the duration of the infusion procedure, while simultaneously limiting the potential for movement.

### Infusion Systems

Also provided herein are otic infusion systems 500 including an otic catheter assembly 200 and a catheter introducer 270. Such catheter assemblies and catheter introducers are described relative to, e.g., FIGS. 14A-14B and 15A-15C. For example, according to certain embodiments of the disclosure, an otic infusion system 500 includes an otic catheter assembly 200 as described herein, and a catheter introducer 270 adapted for, e.g., intracochlear insertion into the RWM. The catheter introducer 270 is adapted to receive the catheter assembly 200 therein, and the catheter introducer 270 and the catheter assembly 200 are adapted to be removed from the RWM together, e.g., as a single device. As described further herein the catheter introducer 270 comprises a depth stop 272 disposed thereon to limit an extent of insertion into, e.g., the RWM.

Also provided herein are otic infusion systems 600 including an otic catheter assembly 200 or other delivery device as described elsewhere herein, and a pump 602 (FIG. 21C) configured to generate the flow of drug product through the catheter assembly 200 to infuse into the subject. The pump 602 may include, e.g., an electroosmotic (EO) pump, a gas-cylinder powered device, a pressure-based device in which lubricant flows through an orifice at a controlled rate, an electrochemical cell, or a battery-driven motor, e.g., a self-powered expanding battery actuator.

### Kits of Parts

Also disclosed herein are kits of parts for performing an otic infusion, e.g., an intracochlear infusion. The kits of parts may include an otic catheter assembly 200 or other delivery device as described herein, an extraction needle (e.g., an 18 G×1½ in. needle), a syringe (e.g., a 1 mL or 3 mL luer lock syringe), and a syringe cap, each of which may be provided in its original sterile package. The kit of parts may also include instructions for use of the kit components with one or more commercially available syringe pumps. The instructions may additionally reference a separate package, apart from the kit of parts, which may contain the drug product. The drug product may be packaged separately from the balance of the kit components due to, e.g., distinct storage requirements of the drug product, e.g., a requirement of ultra-cold storage, and may be separately commercially available. Additionally, certain embodiments of kits of parts may include an instrument, e.g., for forming a hole in the RWM as also described herein. The instrument may be integrated with the catheter assembly (FIG. 19, integrated pick 316) or may be a standalone instrument (FIGS. 9A, 9D, instrument 240). The kit of parts may in further embodiments also include additional components as described elsewhere herein, such as pumps, connectors such as, e.g., luer connections, feed devices, and so on.

### Drug Product and Parts for Independent Sourcing

Also disclosed herein is a drug product, such as those described herein, e.g., DB-OTO, provided in a sterile container such as, e.g., a vial, and packaged together with instructions for use, and a list of parts to be independently sourced for use in methods of administration as described herein. The list of parts may include, e.g., an extraction needle (e.g., an 18 G×1½ in. needle), a syringe (e.g., a 1 mL or 3 mL luer lock syringe), a syringe cap, a delivery device such as, e.g., catheter assembly 200, 700, 800 as described herein, or other delivery device, as well as one or more commercially available syringe pumps which may be used in conjunction with the listed components to carry out the methods of administration described herein.

### Combination Products

Also disclosed herein are combination products which include a therapeutic drug product (DP) and a medical device, e.g., a delivery device as described herein. Non-limiting exemplary DPs which may be included in a combination product as described herein include, e.g., gene therapies such as DB-OTO, polynucleotides and vectors capable of promoting expression of GJB2, polynucleotides and vector systems capable of promoting expression of stereocilin, polynucleotides and vectors capable of increasing expression of the SLC26A4 gene, and small molecule DPs including, e.g., DB-020, as described herein above.

In certain embodiments, the combination product may be a co-packaged combination product, and may include a delivery device packaged together with a DP intended to be administered using the delivery device. The DP may be provided in, e.g., a vial or other sterile packaging.

In certain embodiments, the combination product may be an integrated combination product, in which the DP and the delivery device form a single product. Exemplary integrated combination products may include, e.g., pre-filled syringes having the DP contained within a barrel thereof, and may further include, e.g., 1 mL, 3 mL, or other sized luer lock syringes.

In other embodiments, the combination product may be a referenced combination product, in which the DP and the delivery device are provided as separate products, with the DP being labeled for use with the specific delivery device. The DP and the delivery device may also in certain cases be cross-labeled.

Delivery devices in the context of combination products described herein may include, e.g., catheter assemblies such as catheter assemblies 200, 700, 800, which may be useful for otic administration, e.g. intracochlear administration, of DPs. The combination products may also include accessory kits including, e.g., an extraction needle (e.g., an 18 G×1½ in. needle) for removing the DP from a vial, a syringe (e.g., a 1 mL or 3 mL luer lock syringe) for administering the DP via the delivery device, and a syringe cap, each of which may be provided in its original sterile package. In other embodiments, the combination products described herein may be labeled for use with one or more specific accessory device(s), e.g., needles, syringes, caps, etc. The combination product may additionally be labeled for use with one or more commercially available syringe pumps.

### Embodiments

Embodiments of the present disclosure may include the following features:
Item 1. An intracochlear catheter assembly comprising: a sheath having a first lumen axially extending therethrough; and a distal tip adapted for insertion through the round window membrane (RWM), wherein the catheter assembly is configured for intracochlear insertion.
Item 2. The intracochlear catheter assembly of item 1, further comprising an overcoating disposed over the sheath, such that the sheath forms a liner within the overcoating.
Item 3. The intracochlear catheter assembly of item 2, further comprising: a rigid layer disposed about the liner and within the overcoating, the rigid layer being adapted to provide structural support to the catheter assembly, wherein the rigid layer comprises a metal coil, a metal mesh, or a metal braid.
Item 4. The intracochlear catheter assembly of item 3, further comprising: a needle integrated into the distal tip and in fluid communication with the first lumen, wherein the needle is adapted for insertion through the RWM and infusion of a fluid.
Item 5. The intracochlear catheter assembly of item 1, further comprising an opening at the distal tip of the sheath, the opening being in fluid communication with the first lumen.
Item 6. The intracochlear catheter assembly of item 1, further comprising a tapered geometry at the distal tip of the catheter assembly.
Item 7. The intracochlear catheter assembly of item 1, wherein the distal tip has a shape selected from: blunt, rounded, and pointed.
Item 8. The intracochlear catheter assembly of item 1, further comprising a plurality of through-holes in a radially outward facing surface of the overcoating, wherein each through-hole in the plurality of through-holes is in fluid communication with the first lumen, and wherein the distal end of the first lumen is closed.
Item 9. The intracochlear catheter assembly of item 1, further comprising a connector coupled to a proximal end of the catheter assembly.
Item 10. The intracochlear catheter assembly of item 1, wherein the sheath comprises plastic.
Item 11. The intracochlear catheter assembly of item 2, further comprising a depth stop disposed about the overcoating.
Item 12. The intracochlear catheter assembly of item 11, wherein the depth stop is integrally formed with the overcoating.
Item 13. The intracochlear catheter assembly of item 1, wherein the distal tip is adapted for insertion through a hole in the RWM formed by an instrument comprising a notch or a flange adapted to indicate a desired depth of insertion.
Item 14. The intracochlear catheter assembly of item 1, further comprising a second lumen disposed therein, the second lumen being substantially parallel with the first lumen, and adapted to receive a guidewire or stylet therein.
Item 15. The intracochlear catheter assembly of item 14, wherein the second lumen is open to the proximal end of the catheter assembly and is not open to the distal tip of the catheter assembly.
Item 16. The intracochlear catheter assembly of item 15, wherein the catheter assembly further comprises a tapered portion at the distal tip thereof, and wherein a closed, distal end of the second lumen is disposed proximally relative to a proximal-most end of the tapered portion, such that the second lumen does not extend distally into the tapered portion.
Item 17. The intracochlear catheter assembly of item 15, further comprising a shoulder formed in the overcoating, wherein the shoulder is disposed about a closed distal end of the second lumen, and wherein the shoulder forms a depth stop limiting an extent of insertion of the distal tip of the catheter assembly through the RWM.
Item 18. The intracochlear catheter assembly of item 1, further comprising: a pair of tip members collectively forming the distal tip, each tip member of the pair of tip members being movable between a closed position and an open position, wherein in the closed position, the pair of tip members collectively form a sharp point at a distal end thereof, and the intracochlear catheter assembly is closed at the distal tip, and in the open position, the pair of tip members are adapted to articulate radially outwardly, and the intracochlear catheter assembly is open at the distal tip; and a microcontroller adapted to selectively move the pair of tip members between the closed position and the open position.
Item 19. The intracochlear catheter assembly of item 4, further comprising: a flexible and collapsable housing disposed over the intracochlear catheter assembly, and movable between a flexible, extended state and a rigid, collapsed state, wherein the housing is configured to contain the intracochlear catheter assembly while allowing the needle to extend distally beyond a distal end of the housing, wherein, in the rigid, collapsed state, the housing is configured to secure a position and an orientation of the needle.
Item 20. The intracochlear catheter assembly of item 1, further comprising: an introducer disposed over the intracochlear catheter assembly, the introducer comprising: a sharp distal tip configured to puncture the RWM, and a depth stop disposed proximally relative to the sharp distal tip, the depth stop being configured to limit an extent of insertion of the introducer through the RWM; and a spring disposed within the introducer and adapted to bias the intracochlear catheter assembly in a distal direction.
Item 21. The intracochlear catheter assembly of item 4, further comprising a housing disposed thereover, the housing including: a distal housing member that is disposed over the distal tip of the intracochlear catheter assembly, and configured to limit distal movement thereof upon contact by the distal housing member with the RWM, wherein the needle is permitted to extend distally beyond the distal housing member; and a proximal housing member coupled to the distal housing member, and comprising a flange at a proximal end thereof, the flange being configured to engage an outer surface of a skull of a patient and thereby limit distal movement of the housing relative to the skull.
Item 22. The intracochlear catheter assembly of item 3, further comprising: a first sleeve disposed over the overcoating; a pick disposed within a lumen in the first sleeve and configured to translate distally to puncture the RWM upon actuation; a handle coupled to a proximal end of the first sleeve and having an actuator for actuating translation of the pick; a second sleeve disposed over the first sleeve; and a depth stop disposed on the second sleeve and configured to limit a depth to which the intracochlear catheter assembly is insertable into a patient.
Item 23. A terminally sterilized, pre-filled syringe for intracochlear injection comprising: a barrel having an outlet at an outlet end and containing a medicament; a needle disposed at the outlet end and in fluid communication with the outlet; a stopper disposed and movable within the barrel; a plunger rod in contact with the stopper; a flange disposed at a proximal end of the barrel; and a spacer disposed about the needle at the outlet end, the spacer being configured to limit a depth to which the needle may be inserted in use.
Item 24. An intracochlear infusion system, comprising: an intracochlear catheter assembly comprising: a sheath having a first lumen axially extending therethrough; and a distal tip adapted for insertion through the round window membrane (RWM), wherein the catheter assembly is configured for intracochlear insertion; and a catheter introducer adapted for insertion into the RWM, wherein the catheter introducer is adapted to receive the catheter assembly therein, and wherein the catheter introducer and the catheter assembly are adapted to be removed from the RWM together.
Item 25. The intracochlear infusion system of item 24, wherein the intracochlear catheter assembly further comprises an overcoating disposed over the sheath, such that the sheath acts as a liner within the overcoating.
Item 26. The intracochlear infusion system of item 25, wherein the catheter introducer comprises a further sheath disposed over a portion of the catheter assembly.
Item 27. The intracochlear infusion system of item 25, further comprising: a depth stop coupled to the introducer at a distal end thereof, wherein the distal tip of the catheter assembly extends distally beyond the depth stop.
Item 28. The intracochlear infusion system of item 27, further comprising: a support structure coupled to the depth stop and extending distally therefrom, wherein the support structure includes a sharp distal end configured to puncture the RWM, and wherein the support structure is configured to partially enclose and support the distal tip of the catheter assembly.
Item 29. The intracochlear infusion system of item 27, wherein the support structure comprises a plurality of blades or prongs circumferentially arranged about the distal tip.
Item 30. The intracochlear infusion system of item 27, wherein the support structure comprises a sconce disposed about a portion of a circumference of the distal tip.
Item 31. The intracochlear infusion system of item 27, wherein the support structure comprises a helical structure disposed about the distal tip.
Item 32. The intracochlear infusion system of item 27, further comprising: a depth stop disposed about the catheter assembly; and a plurality of pinch blocks disposed on an inner surface of the catheter introducer, wherein the plurality of pinch blocks are configured to engage the depth stop on the catheter assembly to secure a position of the catheter assembly within the catheter introducer.
Item 33. The intracochlear infusion system of item 32, further comprising: a connector disposed on the depth stop; and an in-lay disposed on a proximal end of the catheter introducer, wherein the in-lay is configured to receive and engage the connector disposed on the depth stop.
Item 34. An intracochlear infusion system, comprising: an intracochlear catheter assembly comprising: a sheath having a first lumen axially extending therethrough; and a distal tip adapted for insertion through a round window membrane (RWM) of a subject, wherein the catheter assembly is configured for intracochlear insertion; and a feed device disposed on a patient's skin at a location proximate to an incision in the skin through which the RWM is accessible, wherein the feed device is configured to receive the intracochlear catheter therethrough.
Item 35. The intracochlear infusion system of item 34, wherein the intracochlear catheter assembly further comprises an overcoating disposed over the sheath.
Item 36. The intracochlear infusion system of item 34, wherein the feed device comprises an adhesive configured to adhere to the patient's skin.
Item 37. The intracochlear infusion system of item 34, wherein the feed device comprises a rotatable dial or knob configured to advance the intracochlear catheter assembly into the RWM, and to retract the intracochlear catheter assembly from the RWM.
Item 38. The intracochlear infusion system of item 37, wherein the rotatable dial or knob is configured to rotate in a first direction to advance the intracochlear catheter assembly into the RWM, and to rotate in a second direction, opposite the first direction, to retract the intracochlear catheter assembly from the RWM
Item 39. The intracochlear infusion system of item 37, wherein the rotatable dial or knob comprises a micro-adjustment knob.
Item 40. The intracochlear infusion system of item 34, wherein the feed device comprises a connector at a proximal end thereof, configured to fluidly couple the intracochlear catheter assembly to an infusion pump.
Item 41. An intracochlear infusion system, comprising: an intracochlear catheter assembly comprising: a sheath having a first lumen axially extending therethrough; and a distal tip adapted for insertion through the round window membrane (RWM), wherein the catheter assembly is configured for intracochlear insertion; and a pump configured to cause fluid to flow through the intracochlear catheter assembly and infuse into a cochlea of a patient.
Item 42. The intracochlear infusion system of item 41, wherein the intracochlear catheter assembly further comprises an overcoating disposed over the liner.
Item 43. The intracochlear infusion system of item 41, wherein the pump comprises an electroosmotic (EO) pump.
Item 44. The intracochlear infusion system of item 41, wherein the pump comprises a gas-cylinder powered device.
Item 45. The intracochlear infusion system of item 41, wherein the pump comprises an electrochemical cell.
Item 46. The intracochlear infusion system of item 41, wherein the pump comprises a battery-driven motor.
Item 47. A kit for performing an intracochlear infusion comprising: an intracochlear catheter assembly comprising: a sheath having a first lumen axially extending therethrough; and a distal tip adapted for insertion through the round window membrane (RWM), wherein the catheter assembly is configured for intracochlear insertion; and an instrument for forming a hole in the RWM, the instrument comprising a depth stop or a depth indicator.
Item 48. The kit of claim 47, wherein the intracochlear catheter assembly further comprises an overcoating disposed over the sheath.
Item 49. The kit of item 47, wherein the instrument comprises a depth stop, and the depth stop comprises a flange.
Item 50. The kit of item 47, wherein the instrument comprises a depth indicator, and the depth indicator comprises a notch.
Item 51. An intracochlear catheter assembly comprising: a microneedle having a sharp distal tip and a first lumen disposed therein; and a catheter having a second lumen, wherein the first lumen and the second lumen are in fluid communication.
Item 52. The intracochlear catheter assembly of item 51, further comprising: a hub coupled to a distal end of the catheter and to a proximal end of the microneedle.
Item 53. The intracochlear catheter assembly of item 52, further comprising: a third lumen extending longitudinally through the hub, wherein the third lumen is adapted to receive the distal end of the catheter and the proximal end of the microneedle.
Item 54. The intracochlear catheter assembly of item 53, further comprising adhesive or a weld adapted to affix a position of the distal end of the catheter within the third lumen.
Item The intracochlear catheter assembly of item 52, wherein the hub is formed about the microneedle by an insert-molding process.
Item 56. The intracochlear catheter assembly of item 55, wherein the microneedle comprises a retention feature disposed at or near a proximal end thereof, wherein the retention feature comprises an outer diameter that is greater than an outer diameter of a shaft of the microneedle.
Item 57. The intracochlear catheter assembly of item 56, wherein the retention feature comprises one of a bead or a flared proximal portion.
Item 58. The intracochlear catheter assembly of item 52, wherein the catheter and the hub each comprise a same material; and a proximal end of the hub and the distal end of the catheter abut one another, and are affixed to one another by a butt weld.
Item 59. The intracochlear catheter assembly of item 52, wherein a proximal end of the hub is disposed within the second lumen of the catheter.
Item 60. The intracochlear catheter assembly of item 59, further comprising a barb disposed on a radially outward facing surface of the hub, wherein the barb contacts a wall of the second lumen.
Item 61. The intracochlear catheter assembly of item 59, further comprising one of: a crimp, a collar, or a clamp disposed about an outer surface of the catheter and configured to apply pressure to a fit between the catheter and the hub.
Item 62. The intracochlear catheter assembly of item 52, wherein, within the hub, the proximal end of the microneedle is disposed within the second lumen at the distal end of the catheter.
Item 63. The intracochlear catheter assembly of item 52, further comprising a depth stop disposed on a distal end of the hub, wherein the depth stop is distanced from the sharp distal tip of the microneedle by a distance corresponding to a desired insertion depth of the microneedle.
Item 64. The intracochlear catheter assembly of item 63, wherein the depth stop comprises a first lobe extending radially outward beyond a circumference of the hub.
Item 65. The intracochlear catheter assembly of item 64, wherein the depth stop further comprises a second lobe extending radially outward beyond the circumference of the hub, wherein the first lobe and the second lobe are separated by cutaway portions, and wherein the cutaway portions are configured to allow a user to visualize the microneedle.
Item 66. The intracochlear catheter assembly of item 65, wherein the depth stop further comprises a third lobe extending radially outward beyond the circumference of the hub, wherein the first lobe, the second lobe, and the third lobe are separated by cutaway portions, and wherein the cutaway portions are configured to allow a user to visualize the microneedle.
Item 67. The intracochlear catheter assembly of item 63, wherein the depth stop further comprises a distal surface that is selected from: flat, convex, or angled.
Item 68. The intracochlear catheter assembly of item 52, wherein the hub comprises an angled portion relative to a longitudinal axis of the hub.
Item 69. The intracochlear catheter assembly of item 51, wherein the microneedle comprises a silicon needle.
Item 70. The intracochlear catheter assembly of item 51, wherein a distal end of the catheter is disposed over a proximal end of the microneedle, and the distal end of the catheter forms a depth stop that is distanced from the sharp distal tip of the microneedle by a distance corresponding to a desired insertion depth of the microneedle.
Item 71. The intracochlear catheter assembly of item 51, wherein the sharp distal tip of the microneedle is selected from: a Quincke needle tip, a Sprotte needle tip, a Whitacre needle tip, a bent needle tip, and a Tuohy needle tip.
Item 72. The intracochlear catheter assembly of item 51, wherein the catheter further comprises a spring configured to bias the catheter in a distal direction.
Item 73. The intracochlear catheter assembly of item 51, further comprising a visible marking on the catheter configured to indicate to a user one of an insertion depth or a position at which to hold the catheter.
Item 74. The intracochlear catheter assembly of item 51, further comprising a luer connection at a proximal end of the catheter, configured to securely couple the catheter to a syringe containing a drug product for infusion.

1. An intracochlear catheter assembly comprising:
a sheath having a first lumen axially extending therethrough; and
a distal tip adapted for insertion through a round window membrane (RWM), wherein the catheter assembly is configured for intracochlear insertion.

2. The intracochlear catheter assembly of claim 1, further comprising an overcoating disposed over at least a portion of the sheath, such that the sheath forms a liner within the overcoating.

3. (canceled)

4. The intracochlear catheter assembly of claim 1, further comprising:
a needle integrated into the distal tip and in fluid communication with the first lumen, wherein the needle is adapted for insertion through the RWM and infusion of a fluid.

5-8. (canceled)

9. The intracochlear catheter assembly of claim 1, further comprising a connector coupled to a proximal end of the catheter assembly.

10. (canceled)

11. The intracochlear catheter assembly of claim 2, further comprising a depth stop disposed about the overcoating.

12. The intracochlear catheter assembly of claim 11, wherein the depth stop is integrally formed with the overcoating.

13. The intracochlear catheter assembly of claim 1, wherein the distal tip is adapted for insertion through a hole in the RWM formed by an instrument comprising a notch or a flange adapted to indicate a desired depth of insertion.

14-19. (canceled)

20. The intracochlear catheter assembly of claim 1, further comprising:
an introducer disposed over the intracochlear catheter assembly, the introducer comprising:
a sharp distal tip configured to puncture the RWM, and
a depth stop disposed proximally relative to the sharp distal tip,
the depth stop being configured to limit an extent of insertion of the introducer through the RWM; and
a spring disposed within the introducer and adapted to bias the intracochlear catheter assembly in a distal direction.

21-23. (canceled)

24. An intracochlear infusion system, comprising:
an intracochlear catheter assembly comprising:
a sheath having a first lumen axially extending therethrough; and
a distal tip adapted for insertion through a round window membrane (RWM), wherein the catheter assembly is configured for intracochlear insertion; and
a catheter introducer adapted for insertion into the RWM,
wherein the catheter introducer is adapted to receive the catheter assembly therein, and
wherein the catheter introducer and the catheter assembly are adapted to be removed from the RWM together.

25. The intracochlear infusion system of claim 24, wherein the intracochlear catheter assembly further comprises an overcoating disposed at least partially over the sheath, such that the sheath acts as a liner within the overcoating.

26. (canceled)

27. The intracochlear infusion system of claim 25, further comprising:
a depth stop coupled to the introducer at a distal end thereof, wherein the distal tip of the catheter assembly extends distally beyond the depth stop.

28. The intracochlear infusion system of claim 27, further comprising:
a support structure coupled to the depth stop and extending distally therefrom, wherein the support structure includes a sharp distal end configured to puncture the RWM, and wherein the support structure is configured to partially enclose and support the distal tip of the catheter assembly.

29-40. (canceled)

41. The intracochlear infusion system of claim 24, further comprising:
a pump configured to cause fluid to flow through the intracochlear catheter assembly and infuse into a cochlea of a patient.

42. (canceled)

43. The intracochlear infusion system of claim 41, wherein the pump comprises an electroosmotic (EO) pump, a gas-cylinder powered device, an electrochemical cell, or a battery-driven motor.

44-46. (canceled)

47. A kit for performing an intracochlear infusion comprising:
an intracochlear catheter assembly comprising:
a sheath having a first lumen axially extending therethrough; and
a distal tip adapted for insertion through a round window membrane (RWM), wherein the catheter assembly is configured for intracochlear insertion; and
an instrument for forming a hole in the RWM, the instrument comprising a depth stop or a depth indicator.

48. The kit of claim 47, wherein the intracochlear catheter assembly further comprises an overcoating disposed at least partially over the sheath.

49. The kit of claim 47, wherein the instrument comprises a depth stop, and the depth stop comprises a flange.

50. The kit of claim 47, wherein the instrument comprises a depth indicator, and the depth indicator comprises a notch.

51-74. (canceled)

75. The kit of claim 47, further comprising an extraction needle, a syringe, and a pump.

76. The kit of claim 75, further comprising a drug product.
